(19) United States
(12) Patent Application Publication
(10) Pub. No.: US 2009/0010948 A1
(43) Pub. Date: Jan. 8, 2009

(54) ANTI-TUMOR VACCINES DELIVERED BY DENDRITIC CELLS DEVOID OF INTERLEUKIN-10

(75) Inventors: Fang Ping Huang, Oxford (GB); Yu Xiao Chen, Kowloon City (HK); Kwan Man, Hong Kong (HK)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: The University of Hong Kong

(21) Appl. No.: 11/957,146

(22) Filed: Dec. 14, 2007

Related U.S. Application Data
(60) Provisional application No. 60/875,072, filed on Dec. 15, 2006.

(51) Int. Cl. A61K 39/00; C12N 5/00; A61K 35/12; A61P 35/00; A61P 37/04

(52) U.S. Cl. 424/184.1; 435/325; 424/93.21

(57) ABSTRACT

It has been discovered that reducing, inhibiting or preventing the expression of immunosuppressive cytokines or tolergenic agents in antigen presenting cells improves the ability of the antigen presenting cell to promote an immune response. One embodiment provides a genetically engineered antigen presenting cell that has reduced or no expression of IL-10. Preferred antigen presenting cells are dendritic cells. Expression of IL-10 can be inhibited or blocked by genetically engineering the antigen presenting cell to express inhibitory nucleic acids that inhibit or prevent the expression mRNA encoding immunosuppressive cytokines. Inhibitory nucleic acids include siRNA, antisense RNA, antisense DNA, microRNA, and enzymatic nucleic acids that target mRNA encoding immunosuppressive cytokines. Immunosuppressive cytokines include, but are not limited to IL-10, TGF-β, IL-27, IL-35, or combinations thereof. Tolerogenic agents include but are not limited to indoleamine 2,3-dioxygenase.

### CROSS-REFERENCE TO RELATED APPLICATIONS

This application claims benefit of and priority to U.S. Provisional Patent Application No. 60/875,072 filed on Dec. 15, 2006, and which is incorporated herein in its entirety.

### FIELD OF THE INVENTION

Aspects of the invention generally relate to genetically engineered antigen presenting cells and vaccines, more particularly to anti-tumor vaccines delivered by dendritic cells with reduced or inhibited immunosuppressive cytokine expression.

### BACKGROUND OF THE INVENTION

The immune system plays an important role in the fight against tumors (Whiteside, T. L., Semin Cancer Biol., 16(1):3-15 (2006)). Tumors are, however, clones of mutated cells that have arisen from body's own cells. Although the mutations may give rise to the so-called tumor associated antigens (TAA), these newly derived or “altered self” antigens are poor immunogens (Li, G., et al., Curr Pharm Des, 11(27):3501-9 (2005)). Moreover, tumors may evade the immune system by interacting actively with host immune cells to block their functions (Whiteside, T. L., Semin Cancer Biol., 16(1):3-15 (2006); Pardoll, D. Annu Rev Immunol. 807-39 (2003); Igney, F. H. and P. H. Krammer, Cancer Immunol Immunother, 54(11):1127-36 (2005)). This may explain why conventional vaccination approaches have repeatedly failed to induce effective anti-tumor immunity (Rivoltini, L., et al., Expert Opin Biol Ther. 5(4):463-76 (2005).

The dendritic cell (DC)-based tumor vaccine is a newly developed therapeutic approach for cancer treatment. Even though limited success has been achieved so far, it remains as one of the most promising immunological approaches in the battle against cancer (Schuler, G. et al., Curr Opin Immunol., 15(2): p. 138-47 (2003); Dallal, R. M. and M. T. Lotze, Curr Opin Immunol., 12(5):583-8 (2000); Steinman, R. M. and M. Dhodapkar, Int J. Cancer. 94(4):459-73 (2001); Reid, D. C. 112(4):874-87 (2001). It aims to promote and enhance specific immunity to cancer cells within the tumor bearing individuals. By such an approach, DCs are used not only as a vector to deliver tumor antigens, but also as a ‘natural adjuvant’ to boost the vaccine efficiency. However, its use has thus far been limited by the lack of clinically achievable general efficacy and consistency (Bodey, B., et al., Anticancer Res, 20(4), 2665-76 (2000)).

T cells are important in anti-tumor immunity. Activation of naïve T cells in particular is an important step in the initiation of T cell immunity. For their uniquely combined immunobiological properties (Banchereau, J. and R. M. Steinman, Nature, 392 (6673):245-52 (1998)), DCs are believed to be the only cell type capable of activating naïve T cells in vivo. Although the main function of DCs is to present antigens to T-cells, what makes DCs special are their potent ability to act as an immunological adjuvant and their diversified regulatory capacities. Importantly, DCs can provide critical molecules, cytokines or co-stimulatory signals to the T-cells they interact with during activation. However, it has gradually become apparent that DCs are not a homogenous population, and their ability to provide the activation signals can vary vastly between different DC subsets, lineages, maturities or functional status. The types and functional conditions, hence the immunogenic ‘quality’ or nature, of the DC employed are believed to be essential (Dallal, R. M. and M. T. Lotze, Curr Opin Immunol., 12(5):583-8 (2000)). Moreover, DC under some conditions can also exert tolerogenic effects on the immune system.

Therefore, it is an object of the invention to provide improved cell-based vaccines.

It is another object of the invention to provide methods and compositions for treating cancer and infections.

### SUMMARY OF THE INVENTION

It has been discovered that reducing, inhibiting or preventing the expression of immunosuppressive cytokines or tolergenic agents in antigen presenting cells improves the ability of the antigen presenting cell to promote an immune response. One embodiment provides a genetically engineered antigen presenting cell that has reduced or no expression of IL-10. Preferred antigen presenting cells are dendritic cells. Expression of IL-10 can be inhibited or blocked by genetically engineering the antigen presenting cell to express inhibitory nucleic acids that inhibit or prevent the expression of mRNA encoding immunosuppressive cytokines. Inhibitory nucleic acids include siRNA, antisense RNA, antisense DNA, microRNA, and enzymatic nucleic acids that target mRNA encoding immunosuppressive cytokines. Immunosuppressive cytokines also include, but are not limited to, TGF-β, IL-27, IL-35, or combinations thereof. Tolerogenic agents include, but are not limited to, indoleamine 2,3-dioxygenase. Another embodiment provides a recombinant antigen presenting cell in which the gene encoding an immunosuppressive cytokine has been deleted or mutated to prevent expression. In one embodiment, the gene encoding IL-10 is deleted, for example, using homologous recombination.

The genetically modified antigen presenting cells can be loaded with a target antigen or antigenic polypeptide, for example tumor specific antigens, viral antigens, bacterial antigens, or antigenic fragments thereof. Alternatively, the genetically modified antigen presenting cells can be genetically engineered to express a target antigen. The genetically engineered antigen presenting cells can be used as a vaccine to treat or prevent cancer and infections. The recombinant antigen presenting cells can be autologous or heterologous. Another embodiment provides a method for creating a animal model for liver cancer by injecting tumor cells into a mammal via the mammals portal vein.

The key findings represent a breakthrough in cancer immunotherapy which will benefit cancer patients of many types. The highly effective cell-based tumor vaccine delivery system is potentially universally applicable to the treatment of many different types of tumors or cancers.

### BRIEF DESCRIPTION OF THE DRAWINGS

FIGS. 1A and 1B are bar graphs of liver to body ratio of inbred C57BL/6 mice injected with HEPA 1-6 cells (5 million cells per mouse in PBS, Mouse groups 1-3, PV-HCC liver tumor model) or PBS only (Group 4, Normal control). After one week, the mice were treated (i.v.) with either wild type DCs (WtDC, Group 2) or IL-10 deficient DCs (IL-10^{-/-}DC, Group 3) loaded with tumor antigens (TA, HEPA 1-6 cell lysate), or with PBS only as controls (Groups 1, 4). The liver to body and spleen to body ratios are shown in (A) and (B) for each of the corresponding treatment groups (1 to 4) respectively.

FIGS. 2A and 2B are line graphs showing tumor size (diameter, m) versus days after tumor cell implantation in mice, by intra-flank injection of the tumor cells (HEPA 1-6) (IF-HCC extra-hepatic mouse tumor model), followed by treatment with the IL-10^{-/-}DC vaccine or controls.

FIG. 3A are line graphs showing tumor size (volume and diameter, m) versus days after vaccination with IL-10^{-/-}DC, followed by tumor cell implantation (IF-HCC extra-hepatic tumor model). FIG. 3B shows representative FACS profiles showing relative frequencies of the remaining tumor antigen-pulsed (CFSE^{hi}) and un-pulsed control (CFSE^{lo}) target cells detected in the spleen and hepatic lymph node (hLN) of the vaccinated mice. FIG. 3C is bar graph of the percentage of tumor-specific killing (lysis) in the lymphoid organs of the vaccinated mice.

FIGS. 4A and 4B are line graphs of tumor size (diameter, m) and percentage of tumor-free mice versus days after implantation of mouse skin tumor cells (B16) in mice (mouse melanoma tumor model) vaccinated with IL-10^{-/-}DC vaccine or controls.

FIG. 5A is a histogram of DCs generated from mouse bone marrow precursors showing MHC class II, CD11c, CD80, CD86 and CD40 expression on IL-10^{-/-}DC (filled histogram) and WtDC (grey solid line), which were Day 7 DCs generated in the presence of GM-CSF alone (GM DC). FIG. 5B are scatter plots showing the frequency of DCs expressing high MHC class II molecules (MHC II^{hi} DC) as determined by gating on CD11c⁺MHC II^{hi} cells (oval gated region) and expressed as the percentage of total CD11c⁺ cells (GM DC), and the bar histograms also compare the MHC II^{hi} cell frequencies of DCs generated in the presence of GM-CSF alone (GM DC), or a combination of GM-CSF and IL-4 (G4 DC).

FIG. 6A-H shows line graphs of cytokine production versus time for G4 DCs and GM DCs generated from the IL-10^{-/-} and the wild type control (Wt) mice, with (filled symbols) or without (open symbols) LPS stimulation.

FIG. 7A shows line graphs of H³-thymidine incorporation (cpm) versus DC:SPC ratio of allogenic and syngenic T responsiveness to live and killed (mitomycin-C treated) DCs. FIG. 7B are line graphs of H³-thymidine incorporation (cpm) versus different DC:SPC ratios (rIL-10 at 10 ng/ml), or serial concentrations of rIL-10 (ng/ml) (DC:SPC at 1:20).

FIG. 8A shows human IL-10 gene target sequence (SEQ ID NO:1). FIG. 8B shows the primer sequences for the human IL-10 siRNA dicer approach (SEQ ID NOs: 2 and 3).

FIG. 9A shows the human IL-10 gene target sequence (SEQ ID NO: 1) with positions corresponding to the pre-designed siRNA sequences indicated. FIG. 9B shows the sequence of human siRNA pre-designed sense and antisense sequences (SE ID NOs: 4-13) for IL-10.

FIG. 10A shows line graphs of IL-10 (pg/ml) or IL-12p70 (pg/ml) production versus time after stimulation (days) by Day 4, 5, or 6 human blood monocyte-derived DC (MN-DC). FIG. 10B shows line graphs of IL-10 (pg/ml) or IL-12p70 (pg/ml) levels versus dosages of hIL-10 siRNA (nM).

FIG. 11A shows the rat IL-10 nucleic acid sequence (SEQ ID NO: 16), with the intended targeted sequence underlined. FIG. 11B shows the sequences of primers (SEQ ID NO:s 14 and 15) for producing siRNA for rat IL-10 (Dicer approach).

FIG. 12A shows Rat IL-10 gene target sequence (SEQ ID NO: 16) with positions corresponding to the two pre-designed siRNA sequences indicated. FIG. 12B shows the sequences of pre-designed siRNA (SEQ ID NOs: 17-20) for rat IL-10.

FIG. 13 shows line graphs of IL-10 levels (pg/ml) versus time after stimulation (days) of Day 4, 5, and 6 DCs (bone marrow-derived) treated with IL-10 siRNA or controls

FIG. 14A is a bar graph of percentage of rats with lung metastasis in the experimental groups after treatment with the IL-10 siRNA-treated DC vaccine or controls. FIG. 14B is a bar graph of spleen/body weight ratio in rats treated with the IL-10 siRNA DC vaccine or controls. FIG. 14C is a bar graph of liver/body weight ratio in rats treated with the IL-10 siRNA DC vaccine or controls. (Rat liver orthotopic tumor lung metastasis model).

FIGS. 15A and 15B show line graphs of tumor size (diameter, mm; and volume, mm³) versus weeks post-tumor implantation in rats vaccinated with IL-10 siRNA-treated DCs loaded with tumor antigens (tumor lysates). (Rat liver orthotopic solid tumor model).

### DETAILED DESCRIPTION OF INVENTION

### I. Definitions

A “vector” is any moiety that is capable of transferring nucleic acid molecules (e.g., polynucleotide or gene sequences) to target cells (e.g., viral vectors, non-viral vectors, particulate carriers, and liposomes). Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors.

“Operably linked” refers to an arrangement of elements wherein the components so described are configured so as to perform their usual function. Thus, a given promoter that is operably linked to a coding sequence (e.g., a sequence encoding an antigen of interest) is capable of effecting the expression of the coding sequence when the regulatory proteins and proper enzymes are present. In some instances, certain control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

“Recombinant” when referring to a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant” when referring to a cell means a cell that has been altered for example to express or inhibit expression of a nucleic acid or to display or present an antigenic molecule.

Techniques for determining nucleic acid and amino acid “sequence identity” also are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman (1981) Advances in Applied Mathematics 2:482 489. This algorithm can be applied to amino acid sequences by using the scoring matrix developed by Dayhoff, Atlas of Protein Sequences and Structure, M. O. Dayhoff ed., 5 suppl. 3:353 358, National Biomedical Research Foundation, Washington, D.C., USA, and normalized by Gribskov (1986) Nucl. Acids Res. 14(6):6745 6763. An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example BLASTN and BLASTP can be used using the following default parameters: genetic code=standard; filter-none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS transtations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80% 85%, preferably at least about 90%, and most preferably at least about 95% 98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. For example, stringent hybridization conditions can include 50% formamide, 5×Denhardt's Solution, 5×SSC, 0.1% SDS and 100.mu.g/ml denatured salmon sperm DNA and the washing conditions can include 2×SSC, 0.1% SDS at 37° C. followed by 1×SSC, 0.1% SDS at 68° C. Defining appropriate hybridization conditions is within the skill of the art. See, e.g., Sambrook et al., supra; DNA Cloning, supra; Nucleic Acid Hybridization, supra.

The term “vaccine composition” refers to any pharmaceutical composition containing an antigen (as used herein the term refers to a composition containing a nucleic acid molecule having a sequence that encodes an antigen), which can be used to prevent or treat a disease or condition in a subject. Vaccine compositions may also contain one or more adjuvants.

An “immunological response” or “immune response” against a selected agent, antigen or a composition of interest refers to a humoral and/or a cellular immune response to molecules (e.g., antigen) present in the agent or composition of interest. A “humoral immune response” refers to an immune response mediated by antibody molecules, while a “cellular immune response” is one mediated by T-lymphocytes and/or other white blood cells.

Mammalian immune responses are understood to involve an immune cascade following one of two broad categories of response, characterized by the class of T helper cell which initiates the cascade. Thus, an immune response to a specific antigen may be characterized as a T helper 1 (Th1)-type or T helper 2 (Th2)-type response, depending on the types of cytokines that are released from antigen-specific T lymphocytes following antigen presentation. Th1 immune responses are generally characterized by the release of inflammatory cytokines, such as IL-2, interferon-gamma (IFN-γ), and tumor necrosis factor alpha (TNF-α), from the antigen-stimulated T helper cells. Th1 responses are also associated with strong cellular immunity (e.g., CTLs) and the production of IgG antibody subclasses that possess opsonizing and complement-fixing activity, such as IgG2a in the commonly used mouse model. On the other hand, Th2 immune responses are characterized by the release of noninflammatory cytokines, such as IL-4 and IL-10, following stimulation of antigen-specific T helper cells. The Th2 responses generally do not favor maximal CTL activity, but are associated with strong antibody responses, representing IgG subclasses such as IgG1 in the mouse, antibody classes that lack opsonizing and complement-fixing activity. In general, the antibody levels associated with Th2 responses are considerably stronger than those associated with Th1 responses.

The term “adjuvant” refers to any material or composition capable of specifically or non-specifically altering, enhancing, directing, redirecting, potentiating or initiating an antigen-specific immune response. Thus, co-administration of an adjuvant and an antigen (e.g., as a vaccine composition) may result in a lower dose or fewer doses of antigen being necessary to achieve a desired immune response in the subject to which the antigen is administered. In certain embodiments, co-administration of an adjuvant with a nucleic acid encoding an antigen can redirect the immune response against the antigen, for example, where the immune response is redirected from a Th2-type to a Th1-type immune response, or vice versa. The effectiveness of an adjuvant can be determined by administering the adjuvant with a vaccine composition and vaccine composition controls (no adjuvant) to animals and comparing antibody titers and/or cellular-mediated immunity against the two using standard assays such as radioimmunoassay, ELISAs, CTL assays, and the like, well known in the art. Typically, in a vaccine composition, the adjuvant is a separate moiety from the antigen, although a single molecule can have both adjuvant and antigen properties (e.g., cholera toxin). An adjuvant can be used to either enhance the immune response to a specific antigen, e.g., when an adjuvant is co-administered with a vaccine composition, the resulting immune response is greater than the immune response elicited by an equivalent amount of the vaccine composition administered without the adjuvant, or the adjuvant is used to redirect the nature of the immune response. An “effective amount” of an adjuvant will be that amount which enhances an immunological response to a co-administered antigen in a vaccine composition such that lower or fewer doses of the antigen are required to generate an efficient immune response. An “effective amount” of an adjuvant will be that amount which is sufficient to bring about a shift or redirection of the immune response relative to the immune response to the antigen alone. An “adjuvant composition” intends any pharmaceutical composition containing an adjuvant.

An “immune shift adjuvant” is an adjuvant that is effective to alter or direct (re-direct) the nature of an immune response against a selected antigen receiving both the antigen and the immune shift adjuvant. The altering or redirecting is relative to the nature of the immune response that is directed against the antigen in the absence of the immune shift adjuvant. Such adjuvants are used to shift the nature of an immune response elicited against a selected antigen (an antigen encoded by a nucleic acid sequence present in a genetic vaccine composition) to favor a Th1-type response in lieu of a Th2-type response, or to favor a Th2-type response in lieu of a Th1-type response. A number of known adjuvants can be used as immune shift adjuvants including, but not limited to, a monophosphoryl lipid A (MPL) adjuvant. The ability of an adjuvant to serve as an immune shift adjuvant can be determined by assessing the nature of immune responses engendered by administration of the vaccine composition alone, and administration of the vaccine composition with the adjuvant. This assessment can involve a characterization or identification of the types of cytokines that are released from antigen-specific T lymphocytes following antigen presentation in an individual and/or the characterization or identification of the predominant IgG subclasses that are elicited by an antigen/adjuvant combination relative to antigen alone. All of these characterization or identifications are well within the skill of the ordinarily skilled artisan as directed by the present specification.

As used herein, the term “co-administered,” such as when an adjuvant is “co-administered” with a nucleic acid encoding an antigen (e.g., a vaccine composition), refers to either the simultaneous or concurrent administration of adjuvant and antigen, e.g., when the two are present in the same composition or administered in separate compositions at nearly the same time but at different sites, as well as the delivery of adjuvant and antigen in separate compositions at different times. For example, the adjuvant composition may be delivered prior to or subsequent to delivery of the antigen at the same or a different site. The timing between adjuvant and antigen deliveries can range from about several minutes apart, to several hours apart, to several days apart.

As used herein, the term “treatment” includes any of following: the prevention or reduction of infection or reinfection; the reduction or elimination of symptoms; and the reduction or complete elimination of a pathogen. Treatment may be effected prophylactically (prior to infection).

### II. Compositions

One embodiment provides cellular compositions for activating T lymphocytes in vivo or ex vivo to elicit an immune response. The cellular composition includes an antigen-presenting cell (APC) modified to have reduced or no expression of one or more immunosuppressive cytokines. A preferred immunosuppressive cytokine that is downregulated or inhibited is IL-10.

Interleukin 10 (IL-10) is a potent immunosuppressive cytokine produced by a variety of immune cell types including DC. IL-10, secreted by some DC subsets and macrophages, can inhibit T-cell activation, while the DC functional activities are in return tightly regulated by this very cytokine (Enk, A. H., 99(1):8-11 (2005)) In addition, some tumor cells may produce IL-10 directly to suppress host immunity by blocking DC functions (Platsoucas, C. D., et al., Anticancer Res. 23(3A):1969-96 (2003)).

Other immunosuppressive cytokines can also be down-regulated. These include, but are not limited to, TGF-β, IL-27, IL-35, or combinations thereof. The antigen presenting cell can also be genetically engineered to have reduced expression of a tolerogenic agent such as indoleamine 2,3-dioxygenase. The modified APCs or vaccine can optionally include an adjuvant. The modified APC also presents an antigen, for example a tumor specific antigen. In a preferred embodiment, the modified APC is a modified dendritic cell.

A. Antigen Presenting Cells

APCs are highly specialized cells, including macrophages, monocytes, and dendritic cells (DCs), that can process antigens and display their peptide fragments on the cell surface together with molecules required for lymphocyte activation. Generally, however, dendritic cells are superior to other antigen presenting cells for inducing a T lymphocyte mediated response (e.g., a primary immune response). DCs may be classified into subgroups, including, e.g., follicular dendritic cells, Langerhans dendritic cells, and epidermal dendritic cells.

DCs have been shown to be potent simulators of both T helper (Th) and cytotoxic T lymphocyte (CTL) responses. See Schuler et al., 1997, Int. Arch. Allergy Immunol. 112:317-22. In vivo, DCs display antigenic peptides in complexes with MHC class I and MHC class II proteins. The loading of MHC class I molecules usually occurs when cytoplasmic proteins (including proteins that are ultimately transported to the nucleus) are processed and transported into the secretory compartments containing the MHC class I molecules. MHC Class II proteins are normally loaded in vivo following sampling (e.g., by endocytosis) by APCs of the extracellular milieu. DCs migrate to lymphoid organs where they induce proliferation and differentiation of antigen-specific T lymphocytes, i.e., Th cells that recognize the peptide/MHC Class II complex and CTLs that recognize the peptide/MHC Class I complex.

DCs (or DC precursor cells) can be exposed to antigenic peptide fragments ex vivo (referred to as “antigen pulsing”), or genetically modified ex vivo to express a desired antigen, and subsequently administered to a patient to induce an anti-antigen immune response. Alternatively, the pulsed or genetically modified DCs can be cultured ex vivo with T lymphocytes (e.g., HLA-matched T lymphocytes) to activate those T cells that are specific for the selected antigen. Antigen-laden DC may be used to boost host defense against tumors. It will be appreciated that is not necessary that the target antigen (e.g., target “tumor” antigen) be expressed naturally on the cell surface, because cytoplasmic proteins and nuclear proteins are normally processed, attached to MHC-encoded products intracellularly, and translocated to the cell surface as a peptide/MHC complex.

In one aspect, polypeptides and/or polynucleotides encoding target antigens, and antigen presenting cells (especially dendritic cells), are used to elicit an immune response against cells expressing or displaying the target antigen, such as cancer cells, in a subject. Typically, this involves (1) isolating hematopoietic stem cells, (2) genetically modifying the cells to express the target antigen and to inhibit expression of one or more immunosuppressive cytokines, (3) differentiating the precursor cells into DCs and (4) administering the DCs to the subject (e.g., human patient). In an alternative embodiment, the process involves (1) isolating DCs (or isolation and differentiation of DC precursor cells) (2) genetically modifying the cells to inhibit expression of one or more immunosuppressive cytokines (3) pulsing the cells with target antigen, and (4) administering the DCs to the subject. In another embodiment, the antigen pulsed or antigen expressing DCs are used to activate T lymphocytes ex vivo.

1. Genetic Modification of Dendritic Cell Precursors

In one embodiment, DC progenitor cells are isolated to genetically downregulate, reduce or block expression of one or more immunosuppressive cytokines, preferably IL-10. The modified DC progenitor cells are then induced to differentiate into dendritic cells. Optionally, the DC progenitor cells can be genetically altered to express or overexpress a target antigen. The genetically modified DCs have no or reduced expression of IL-10 relative to a control, for example an unmodified DC or DC progenitor cell. The modified DCs display peptide fragments of the target antigen on the cell surface.

Many methods are known for isolating DC precursor cells suitable for genetic manipulation to downregulate cytokine expression and to optionally express a target antigen. Human hematopoietic progenitor and stem cells are characterized by the presence of a CD34 surface membrane antigen, which may be used in purification. In one embodiment, for example, human hematopoietic stem cells are obtained by bone marrow aspiration, and the bone marrow mononuclear cells are separated from the other components by means of Ficol density gradient centrifugation and adherence to plastic. The light density, non-adherent cells are obtained and further selected using an anti-CD34 antibody (preferably monoclonal) by standard methods (e.g., incubation of cells with the anti-CD34 antibody, subsequent binding to an immobilized secondary antibody, and removal of nonbound components; see, e.g., Harlow and Lane, 1988, ANTIBODIES: A LABORATORY MANUAL, Cold Spring Harbor Laboratory, New York) Alternatively, cells can be obtained by leukapheresis of peripheral blood and anti-CD34 chromatography (see, e.g., Reeves et al, 1996, Cancer Res. 56:5672-77).

In one embodiment, the DC or DC precursor cell is genetically modified to down regulate expression of one or more immunosuppressive cytokines such as IL-10. Downregulation of IL-10 can be achieved using well known techniques including, but not limited to, genetically modifying the DC to expressing antisense DNA, siRNA, microRNA, or an enzymatic nucleic acid that is specific for mRNA encoding IL-10.

The DC or DC precursor can be genetically modified to express a target antigen polypeptide (e.g., transduced ex vivo with a polynucleotide encoding the target antigen polypeptide). Exogenous antigen-encoding polynucleotides or polynucleotides that down regulate IL-10 mRNA expression may be incorporated into DC as expression cassettes using methods known in the art. Typically the DC is transformed with an expression cassette comprising a region encoding a target antigen polypeptide (or one or more antigenic fragments thereof) or polynucleotides that down regulate IL-10 mRNA expression. Upon expression of the expression cassette in the cell, the target antigen polypeptide is processed into antigenic peptides expressed on the surface of the DC as a complex with MHC class I and II surface molecules.

Typically the expression cassette includes an operably linked promoter (to drive expression of the antigen coding sequences). Usually a strong promoter such as a t-RNA pol III promoter or a pol II promoter with strong constitutive expression is used. Suitable promoters include the constitutive adenovirus major late promoter, the dexamethasone-inducible MMIV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art. In alternative embodiments, the antigen coding sequence is introduced into the DC precursor without a linked promoter. In such a case transcription is directed by an endogenous promoter (e.g., following integration of the antigen coding sequence into the cell chromosome) or a separately introduced promoter (e.g., that becomes linked by recombination). Often the expression cassette is contained in an expression vector such as a plasmid or viral vector, which may also include other elements, e.g., an origin of replication, chromosome integration elements such as retroviral LTRs, and/or selection (e.g., drug resistance) sequences.

In one embodiment all or of most (e.g., at least about 60%, at least about 75% or at least about 90%) of the antigen protein is expressed (i.e., coded for) in the antigen expression cassette. In some cases, however, a shorter fragment may be expressed. Usually antigen coding sequence will encode at least about 8, more often 12, still more often at least 30 or at least 50 contiguous antigen amino acid residues.

The expression sequence may be introduced (transduced) into DCs or stem cells in any of a variety of standard methods, including transfection, recombinant vaccinia viruses, adeno-associated viruses (AAVs), and retroviruses, particle-mediated gene transfer technology, or other conventional methods for transforming stem cells are known. Alternately, polynucleotides can be packaged into viral particles using packaging cell lines, which are incubated with the DC stem cells.

The recombinant hematopoietic progenitor cells described above are induced to differentiate into DCs by conventional methods, e.g., by exposure to cytokines such as granulocyte macrophage colony-stimulating factor (GM-CSF), flt-3 ligand, tumor necrosis factor alpha c-kit ligand (also called steel factor or mast cell factor). The addition of interleukin-4 (IL-4) to monocyte cultures is reported to help direct cells to develop as dendritic cells, and TNF-alpha, when mixed with undifferentiated stem cells, increases the likelihood that the stem cells will develop as dendritic cells (see Szaboles et al., J. Immunol. 154:5851-5861 (1995)). Alternatively, calcium ionophore is used to stimulate the maturation of isolated monocytes into dendritic cells (U.S. Pat. No. 5,643,786). In one embodiment, DCs are obtained from CD34+ hematopoietic progenitor cells from the blood (e.g., of cancer patients) according to the method described by Bernhard et al., Cancer Res. 55:1099-104 (1995)). A DC maturation factor may be used to cause “immature DCs” to stably express dendritic cell characteristics (e.g., dendritic cell markers p55 and CD83; see WO 97/29182). Alternatively, immature DCs may be used to activate T cells (Koch et al., 1995, J. Immunol. 155:93-100).

The culture of cells such as stem cells and dendritic cells is well known in the art.

As used herein “stem cells” includes any cell capable of producing a dendritic cell or a dendritic-like cell.

2. Pulsing APCs with Antigens

In one embodiment DCs modified to down-regulate expression of IL-10 are exposed ex vivo to target antigens and allowed to process the antigen so that antigen epitopes are presented on the surface of the cell in the context of a MHC class I (or MHC class II) complex. This procedure is referred to as “antigen pulsing.” The “pulsed DCs” may then be used to activate T lymphocytes.

The peptide antigens used for pulsing DCs includes at least one linear epitope derived from the antigen protein. Antigenic proteins or antigenic fragments thereof may be used, as they will be taken up and processed by the DCs. Alternatively, short “peptides” may be administered to the DCs.

When antigenic peptides are used for pulsing, they will usually have at least about 6 or 8 amino acids and fewer than about 30 amino acids or fewer than about 50 amino acid residues in length. In one embodiment, the immunogenic peptide has between about 8 and 12 amino acids. A mixture of antigenic protein fragments may be used; alternatively a particular peptide of defined sequence may be used. The peptide antigens may be produced by de novo peptide synthesis, enzymatic digestion of purified or recombinant proteins, by purification of antigens from a natural source (e.g., a patient or tumor cells from a patient), or expression of a recombinant polynucleotide encoding a antigen polypeptides.

The amount of antigen used for pulsing DC will depend on the nature, size and purity of the peptide or polypeptide. Typically, from about 0.05 μg/ml to about 1 mg/ml, most often from about 1 to about 100 μg/ml of peptide antigen is used. After adding the peptide antigen(s) to the cultured DC, the cells are then allowed sufficient time to take up and process the antigen and express antigen peptides on the cell surface in association with either class I or class II MHC. Typically this occurs in about 18-30 hours, most often about 24 hours. In one exemplary embodiment enriched DC are resuspended (10⁶ cells/ml) in RPMI media (Gibco) and cultured with (50 μg/ml) peptide antigens overnight under standard conditions (e.g., 37° C. humidified incubator/5% CO2).

The DCs can be collected and administered as described above.

B. Antigens

Antigens or antigenic peptide fragments can be tumor-specific antigens or antigenic fragments thereof including, but are not limited to, any of the various MAGEs (melanoma associated antigen E), including MAGE 1, MAGE 2, MAGE 3 (HLA-A1 peptide), MAGE 4, etc.; any of the various tyrosinases (HLA-A2 peptide); mutant ras; mutant p53; and p97 melanoma antigen. Other tumor-specific antigens include the Ras peptide and p53 peptide associated with advanced cancers, the HPV 16/18 and E6/E7 antigens associated with cervical cancers, MUC1-KLH antigen associated with breast carcinoma, CA125 and OCAA antigens associated with ovarian cancer, CEA (carcinoembryonic antigen) associated with colorectal cancer, gp100 or MARTI antigens associated with melanoma, and the PSA antigen associated with prostate cancer. The p53 gene sequence is known (see e.g., Harris et al. Mol. Cell. Biol. 6:4650 4656 (1986)) and is deposited with GenBank under Accession No. M14694.

Suitable viral antigens include, but are not limited to, antigens obtained or derived from the hepatitis family of viruses, including hepatitis A virus (HAV), hepatitis B virus (HBV), hepatitis C virus (HCV), the delta hepatitis virus (HDV), hepatitis E virus (HEV) and hepatitis G virus (HGV). By way of example, the viral genomic sequence of HBV is known, as are methods for obtaining antigen-encoding sequences therefrom. See, e.g., Ganem et al. Annu. Rev. Biochem. 56:651 693 (1987); Hollinger, F. B. Hepatitis B virus, vol. II, pp. 2171 2235 (1990), in Fields et al. (eds), Virology, 2nd ed, Raven Press, New York, N.Y.; and Valenzuela et al. (1980) The nucleotide Sequence of the Hepatitis B viral Genome and the Identification of the Major Viral Genes, pp. 57 70, in Fields et al. (eds), Animal Virus Genetics, Academic Press, New York, N.Y.). The HBV genome encodes several viral proteins, including the large, middle and major surface antigen polypeptides, the X-gene polypeptide, and the core polypeptide. In like manner, the viral genomic sequence of HCV is known, as are methods for obtaining the sequence. See, e.g., International Publication Nos. WO 89/04669; WO 90/11089; and WO 90/14436. The HCV genome encodes several viral proteins, including E1 and E2. See, e.g., Houghton et al.) Hepatology 14:381 388 (1991. The sequences encoding these HBV and HCV proteins, as well as antigenic fragments thereof, can be used. Similarly, the coding sequence for the Δ-antigen from HDV is known (see, e.g., U.S. Pat. No. 5,378,814).

The antigen can be from a wide variety of protein antigens from the herpesvirus family, including antigens derived or obtained from herpes simplex virus (HSV) types 1 and 2, such as HSV-1 and HSV-2 glycoproteins gB, gD and gH; antigens from varicella zoster virus (VZV), Epstein-Barr virus (EBV) and cytomegalovirus (CMV) including CMV gB and gH; and antigens from other human herpesviruses such as HHV6 and HHV7. These sequences are also known in the art.

Antigens derived or obtained from other viruses, including, but not limited to, the families Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Bunyaviridae; Arenaviridae; Retroviradae (e.g., HTLV-I; HTLV-II; HIV-1 (also known as HTLV-III, LAV, ARV, hTLR, etc.)), including but not limited to antigens from the isolates HIV.sub.IIB, HIV_{SF2}, HIV_{LAV}, HIV_{LAI}, HIV_{MN}); HIV-1_{CM235}, HIV-1_{US4}; HIV-2, among others. See, e.g. Virology, 3rd Edition (W. K. Joklik ed. 1988); Fundamental Virology, 2nd Edition (B. N. Fields and D. M. Knipe, eds. 1991), for a description of these and other viruses. HIV antigens, such as the gp120 sequences for a multitude of HIV-1 and HIV-2 isolates, including members of the various genetic subtypes of HIV, are known and reported (see, e.g., Myers et al., Los Alamos Database, Los Alamos National Laboratory, Los Alamos, N. Mex. (1992); and Modrow et al. J. Virol. 61:570 578 (1987)) and antigens derived from any of these isolates or other immunogenic moieties derived from any of the various HIV isolates, including any of the various envelope proteins such as gp160 and gp41, gag antigens such as p24gag and p55gag, as well as proteins derived from the pol, env, tat, vif rev, nef vpr, vpu and LTR regions of HIV, may be used.

Suitable bacterial and parasitic antigens are obtained or derived from known causative agents responsible for diseases such as Diptheria, Pertussis, Tetanus, Tuberculosis, Bacterial or Fungal Pneumonia, Cholera, Typhoid, Plague, Shigellosis or Salmonellosis, Legionaire's Disease, Lyme Disease, Leprosy, Malaria, Hookworm, Onchocerciasis, Schistosomiasis, Trypamasomialsis, Lesmaniasis, Giardia, Amoebiasis, Filariasis, Borrelia, and Trichinosis. Still further antigens can be obtained or derived from unconventional viruses or virus-like agents such as the causative agents of kuru, Creutzfeldt-Jakob disease (CJD), scrapie, transmissible mink encephalopathy, and chronic wasting diseases, or from proteinaceous infectious particles such as prions that are associated with mad cow disease.

Suitable allergens include, but are not limited to, allergens from pollens, animal dander, grasses, molds, dusts, antibiotics, stinging insect venoms, and a variety of environmental, drug and food allergens. Common tree allergens include pollens from cottonwood, popular, ash, birch, maple, oak, elm, hickory, and pecan trees; common plant allergens include those from rye, ragweed, English plantain, sorrel-dock and pigweed; plant contact allergens include those from poison oak, poison ivy and nettles; common grass allergens include Timothy, Johnson, Bermuda, fescue and bluegrass allergens; common allergens can also be obtained from molds or fungi such as Alternaria, Fusarium, Hormodendrum, Aspergillus, Micropolyspora, Mucor and thermophilic actinomycetes; penicillin and tetracycline are common antibiotic allergens; epidermal allergens can be obtained from house or organic dusts (typically fungal in origin), from insects such as house mites (dermalphagoides pterosinyssis), or from animal sources such as feathers, and cat and dog dander; common food allergens include milk and cheese (diary), egg, wheat, nut (e.g., peanut), seafood (e.g., shellfish), pea, bean and gluten allergens; common drug allergens include local anesthetic and salicylate allergens; antibiotic allergens include penicillin and sulfonamide allergens; and common insect allergens include bee, wasp and ant venom, and cockroach calyx allergens. Particularly well characterized allergens include, but are not limited to, the major and cryptic epitopes of the Der p I allergen (Hoyne et al. Immunology 83190 195 (1994)), bee venom phospholipase A2 (PLA) (Akdis et al. J. Clin. Invest. 98:1676 1683 (1996)), birch pollen allergen Bet v 1 (Bauer et al. Clin. Exp. Immunol. 107:536 541 (1997)), and the multi-epitopic recombinant grass allergen rKBG8.3 (Cao et al. Immunology 90:46 51 (1997)). These and other suitable allergens are commercially available and/or can be readily prepared following known techniques.

The coding sequence for the antigen of interest can be obtained and/or prepared using known methods. For example, substantially pure antigen preparations can be obtained using standard molecular biological tools. That is, polynucleotide sequences coding for the above-described antigens can be obtained using recombinant methods, such as by screening cDNA and genomic libraries from cells expressing the gene, or by deriving the gene from a vector known to include the same. Furthermore, the desired sequence can be isolated directly from cells and tissues containing the same, using standard techniques, such as phenol extraction and PCR of cDNA or genomic DNA. See, e.g., Sambrook et al., supra, for a description of techniques used to obtain and isolate DNA. Polynucleotide sequences can also be produced synthetically, rather than cloned.

C. Adjuvant

The vaccine compositions optionally include an adjuvant. The adjuvant component can be any suitable adjuvant or combination of adjuvants. For example, suitable adjuvants include, without limitation, adjuvants formed from aluminum salts (alum), such as aluminum hydroxide, aluminum phosphate, aluminum sulfate, etc; oil-in-water and water-in-oil emulsion formulations, such as Complete Freunds Adjuvants (CFA) and Incomplete Freunds Adjuvant (IFA); mineral gels; block copolymers; Avridine™ lipid-amine; SEAM62; adjuvants formed from bacterial cell wall components such as adjuvants including lipopolysaccharides (e.g., lipid A or monophosphoryl lipid A (MPL), trehalose dimycolate (TDM), and cell wall skeleton (CWS); heat shock protein or derivatives thereof; adjuvants derived from ADP-ribosylating bacterial toxins, including diphtheria toxin (DT), pertussis toxin (PT), cholera toxin (CT), the E. coli heat-labile toxins (LT1 and LT2), Pseudomonas endotoxin A, Pseudomonas exotoxin S, B. cereus exoenzyme, B. sphaericus toxin, C. botulinum C2 and C3 toxins, C. limosum exoenzyme, as well as toxins from C. perfringens, C. spiriforma and C. difficile, staphylococcus aureus EDIN, and ADP-ribosylating bacterial toxin mutants such as CRM197, a non-toxic diphtheria toxin mutant; saponin adjuvants such as Quil A (U.S. Pat. No. 5,057,540), or particles generated from saponins such as ISCOMs (immunostimulating complexes); chemokines and cytokines, such as interleukins (e.g., IL-1 L-2, IL-4, IL-5, IL-6, IL-7, IL-S, IL-12, etc.), interferons (e.g., gamma interferon), macrophage colony stimulating factor (M-CSF), tumor necrosis factor (TNF), defensins 1 or 2, RANTES, MIP1-.alpha. and MIP-2, etc; muramyl peptides such as N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-s-n-glycero-3 huydroxyphosphoryloxy)-ethylamine (MTP-PE) etc; adjuvants derived from the CpG family of molecules, CpG dinucleotides and synthetic oligonucleotides which comprise CpG motifs, limosum exoenzyme and synthetic adjuvants such as PCPP (Poly[di(carboxylatophenoxy)phosphazene). Such adjuvants are commercially available from a number of distributors such as, Accurate Chemicals; Ribi Immunechemicals, Hamilton, Mont.; GIBCO; Sigma, St. Louis, Mo.

Several adjuvants are preferred for use as an immune shift adjuvant. An important attribute of such an adjuvant is that it tends to redirect the elicited immune response in a particular desired direction relative to use of the antigen being presented. It is particularly desirable if the adjuvant has the attribute of directing or shifting the immune response toward Th1 as opposed to Th2 responses. Since all immune responses to an antigen are complex, and many if not all immune responses involve elements of both Th1 and Th2 responses, it is not practical to seek total response re-direction. Instead, what is contemplated is a relative shift of type of immune response, for example, by using an adjuvant to enhance a Th1 type response. For example, where it has been found that a particular antigen produces a predominantly Th2 response, and a Th1 response is a more desired outcome, a shift in the direction of Th1 will show greater clinical efficacy from the vaccine. An immune shift adjuvant as described herein may or may not result in any increase in the total quantitative immune response in the individual, which is the result usually sought by the incorporation of adjuvants in vaccines. Instead, the immune shift adjuvant is intended to shift or re-direct the nature or quality of the immune response rather than its magnitude or quantity.

An example of an immune shift adjuvant which favors the Th1 response is monophosphoryl lipid A, or MPL available from Ribi Immunochemical Research, Inc. An example of an immune shift adjuvant which favors the Th2 response is 1,25-dihydroxy vitamin D₃. Other possible immune shift adjuvants include PPD, a purified protein derivative of Bacillus calmette guerin (BCG), trehalose dimycolate, and mycobacterial cell wall skeletal material.

The adjuvant may be present in the instant compositions individually or in a combination of two or more adjuvants. Combined adjuvants may have an additive or a synergistic effect in promoting or shifting an immune response. A synergistic effect is one where the result achieved by combining two or more adjuvants is greater than one would expect than by merely adding the result achieved with each adjuvant when administered individually.

Unfortunately, a majority of the above-referenced adjuvants are known to be highly toxic, and are thus generally considered too toxic for human use. It is for this reason that the only adjuvant currently approved for human usage is alum, an aluminum salt composition. Nevertheless, a number of the above adjuvants are commonly used in animals and thus suitable for numerous intended subjects, and several are undergoing preclinical and clinical studies for human use. However, it has been found that adjuvants which are generally considered too toxic for human use may be administered with a powder injection technique (such as the preferred particle-mediated delivery technique used herein) without concomitant toxicity problems. Without being bound by a particular theory, it appears that delivery of small amounts of adjuvants to the skin allows interaction with Langerhans cells in the epidermal layer and dendritic cells in the cutaneous layer of the skin. These cells are important in initiation and maintenance of an immune response. Thus, an enhanced adjuvant effect can be obtained by targeting delivery into or near such cells. Transdermal delivery of adjuvants may avoid toxicity problems because (1) the top layers of the skin are poorly vascularized, thus the amount of adjuvant entering the systemic circulation is reduced which reduces the toxic effect; (2) skin cells are constantly being sloughed, therefore residual adjuvant is eliminated rather than absorbed; and (3) substantially less adjuvant can be administered to produce a suitable adjuvant effect (as compared with adjuvant that is delivered using conventional techniques such as intramuscular injection).

Once selected, one or more adjuvant can be provided in a suitable pharmaceutical form for parenteral delivery, the preparation of which forms are well within the general skill of the art. See, e.g., Remington's Pharmaceutical Sciences (1990) Mack Publishing Company, Easton, Pa., 18th edition. Alternatively, the adjuvant can be rendered into particulate form as described in detail below. The adjuvant(s) will be present in the pharmaceutical form in an amount sufficient to bring about the desired effect, that is, either to enhance the mucosal response against the co-administered antigen of interest, and/or to direct a mucosal immune response against the antigen of interest. Generally about 0.1 μg to 1000 μg of adjuvant, more preferably about 1 μg to 500 μg of adjuvant, and more preferably about 5 μg to 300 μg of adjuvant will be effective to enhance an immune response of a given antigen. Thus, for example, for Quil A, doses in the range of about 0.5 to 50 μg, preferably about 1 to 25 μg, and more preferably about 5 to 20 μg, will find use with the present methods. For MPL, a dose in the range of about 1 to 250 μg, preferably about 20 to 150 μg, and more preferably about 40 to 75 μg, will find use with the present methods.

Doses for other adjuvants can readily be determined by one of skill in the art using routine methods. The amount to administer will depend on a number of factors including the co-administered antigen, as well as the ability of the adjuvant to act as stimulator of an immune response or to act as an immune shift adjuvant.

### III. Methods of Treatment

The recombinant DCs are introduced into the subject (e.g., human patient) where they induce an immune response. Typically the immune response includes a CTL response against target cells bearing target antigenic peptides (e.g., in a MHC class I/peptide complex). These target cells are typically cancer cells.

When the modified DCs are to be administered to a patient they are preferably isolated or derived from precursor cells from that patient (i.e., the DCs are administered to an autologous patient). However, the cells may be infused into HLA-matched allogeneic, or HLA-mismatched allogenic patients. In the latter case, immunosuppressive drugs may be administered to the recipient.

The cells are administered in any suitable manner, preferably with a pharmaceutically acceptable carrier (e.g., saline). Usually administration will be intravenous, but intra-articular, intramuscular, intradermal, intraperitoneal, and subcutaneous routes are also acceptable. Administration (i.e., immunization) may be repeated at time intervals. Infusions of DC may be combined with administration of cytokines that act to maintain DC number and activity (e.g., GM-CSF, IL-12).

The dose administered to a patient should be sufficient to induce an immune response as detected by assays which measure T cell proliferation, T lymphocyte cytotoxicity, and/or effect a beneficial therapeutic response in the patient over time, e.g., to inhibit growth of cancer cells or result in reduction in the number of cancer cells or the size of a tumor. Typically, 10⁶ to 10⁹ or more DCs are infused, if available.

A. Methods for Treating Cancer

The disclosed modified antigen presenting cells can be used to treat uncontrolled cell division or cancers including, but not limited to, leukemia, lymphoma, gynecological cancers, lung cancer, gastric esophageal cancer, intestinal and colorectal cancer, pancreatic cancer, as well as tumors of kidney and bladder etc. The antigen presenting cells can be autologous or heterologous and can be administered to a subject or patient in need treatment as described above. Treatment includes reducing or alleviating one or more symptoms associated with a disorder.

In one embodiment, the modified antigen presenting cells are administered to a subject to reduce the size of a tumor, reduce the number of tumors, or to prevent metastasis.

B. Methods for Treating Infection

One embodiment provides a method for treating an infection by administering modified antigen presenting cells to subject wherein the modified antigen presenting cells display bacterial or viral antigens or antigenic peptides. Viral infections that can be treated include, but are not limited to, HIV, influenza, hepatitis, herpes, as well as those viruses listed above.

C. Adjunctive Therapy

The compositions described herein can be used as adjunctive therapy, along with, before and/or after treatment with other conventional therapies. For example, patients with tumors may be treated with cytotoxic drugs such as cisplatin, BCNU, methotrexate, or taxol, antibodies such as Herceptin®, radiation, or cytokines or growth factors, such as an interleukin or GM-CSF. Patients with infection may be treated with antibiotics, antivirals or anti-parasitic drugs. Collectively, treatment with such drugs is referred to herein as “chemotherapy”. Patients may also or alternatively be treated surgically to remove cancerous or infected tissue.

### IV. Non-Human Animal Models

Another embodiment provides a method for inducing tumors in a mammal by administering tumor cells via the portal vein. The mammal is preferably a non-human mammal such as mice, rats, goats, sheep, etc. Suitable tumor cells include but are not limited to HEPA 1-6 cells of mouse, and CRL1601 of rat. Typically about 5 million tumor cells in PBS are injected into a mouse through the portal vein.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### IL-10^{-/-}DC as a Highly Immunogenic Cell Vector for the Delivery of Vaccines Against Hepatocellular Carcinoma in a Mouse Model

Materials and Methods

### (a) The Establishment of a Liver Tumor Model in Immune Competent Mice:

Several mouse models of liver cancer have been previously developed. These models are however established either as a special transgenic animal for a particular liver cancer gene, or in immunodeficient mice (e.g. nude mice with no T-cells). The primary function of DC is to activate T-cells, especially naïve T-cells, important in the initiation of immune responses. To study DC functions and their roles in anti-tumor immunity, therefore, it is necessary to create such a model in immunocompetent animals.

A novel liver cancer model was successfully developed in an immune competent normal C57BL/6 inbred mouse strain, by direct injection of the tumor cells (HEPA 1-6 mouse HCC cell line, of C57BL/6 origin) into liver through the portal vein (PV-HCC).

Under anesthesia and at laparectomy, groups of normal inbred C57BL/6 mice were, under anesthesia and at laparectomy, injected through the portal vein with different numbers of HEPA 1-6 cells (1, 2.5, 5 and 10 million cells/mouse in PBS) or PBS alone (control), and the mice were sacrificed at 1, 2, 3 and 4 weeks post-injection of the tumor cells.

Results

Hepatomegaly and splenomegaly were observed in the HEPA 1-6 cell-injected mouse group. Histo-pathological examination confirmed the development of numerous tumor nodules in the livers of HEPA 1-6 cells (HEPA 1-6), but not PBS (control), injected mice. The portal vein model of liver cancer (PV-HCC) was subsequently used to evaluate efficacy of the novel tumor vaccines.

### (b) A Superior Therapeutic IL-10^{-/-}DC Vaccine Against Liver Cancer in the PV-HCC Mouse Model.

The immunogenecity and efficacy of IL-10^{-/-}DC as the vector to deliver tumor vaccine was evaluated in the established liver cancer model mentioned above. DCs devoid of IL-10 are found to be superior to conventional DCs (WtDC) for their ability in promoting anti-tumor immunity that effectively blocks tumor development.

Materials and Methods

Groups of normal inbred C57BL/6 mice were injected through the portal vein with live HEPA 1-6 cells (5 million cells per mouse in PBS, Mouse groups 1-3) or PBS only control (Group 4, Normal control). After one week, the mice were treated (i.v. single injection) with either wild type DCs (WtDC, Group 2) or IL-1 deficient DCs (IL-10-DC, Group 3) loaded in vitro with tumor antigens (TA, HEPA 1-6 cell lysate), or with PBS only as controls (Groups 1, 4). The mice were sacrificed at Week-2.

Results

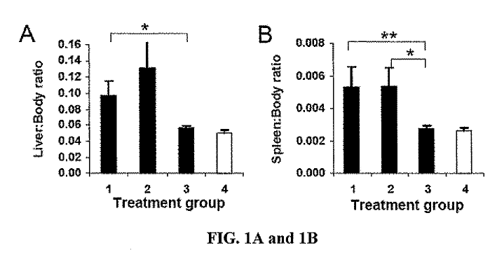

Hepatomegaly and splenomegaly were observed grossly in the untreated (Group 1) and the WtDC/TA treated (Group 2) mice, but not the IL-10−/−DC/TA treated mice (Group 3) or the normal control mice (Group 4). The liver to body and spleen to body ratios are shown in FIGS. 1A and 1B for each of the corresponding treatment groups (1 to 4) respectively. Histopathological examination of the livers revealed numerous tumor nodules (TN) developed in livers of the untreated (Group 1) and the WtDC/TA treated (Group 2) mice, but not the IL-10^{-/-}DC/TA treated (Group 3) or the normal control (Group 4) mice.

### Example 2

### Therapeutic Potential of the IL-10−/−DC Vaccines Reconfirmed In an Intra-Flank HCC Tumor Model

To confirm the therapeutic potential, the DC tumor vaccine was also tested systemically in an extra-hepatic tumor model, which was established by intra-flank subcutaneous injection of the tumor cells in immunocompetent mice (IF-HCC).

Materials and Method

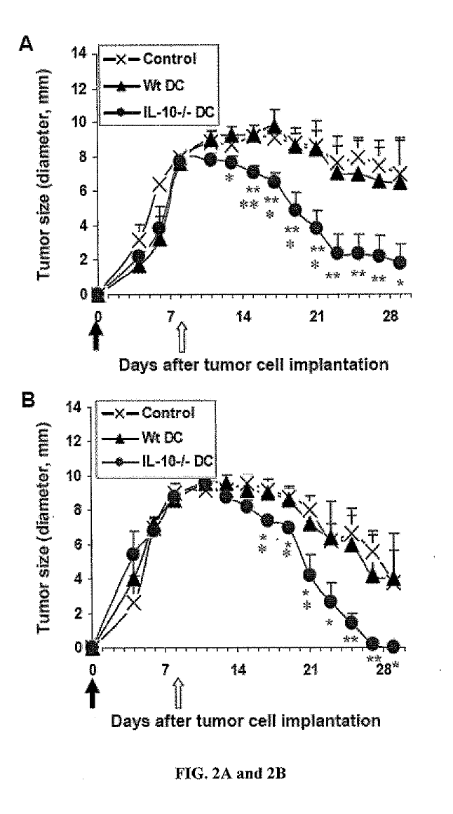

Normal C57BL/6 mice were injected into the left flank s.c. with 10-million Hepa 1-6 tumor cells at Day 0 (solid arrow). By Day 7, sizable tumors were measurable (7-10 mm in diameter) and, at Day 8 (open arrow), the mice were given intravenously one injection of the tumor antigen-loaded WtDCs or IL-10^{-/-}DCs. Tumor development was measured at daily intervals, and expressed as diameters of the tumor mass. Control groups (Control) were tumor-bearing mice treated with PBS only. DCs generated in the presence of GM-CSF alone (GM DC, results combined from 2 repeated experiments, n=9, FIG. 2A) or GM-CSF plus IL-4 (G4 DC, n=5, FIG. 2B) respectively were compared. *depicts significant differences between the IL-10^{-/-}DC and WtDC vaccine-treated groups, and *depicts significant difference between the IL-10−/−DC vaccine-treated group and the PBS-treated tumor control group (Student's T test, * or *p<0.05, ** or **p<0.01).

Results

The results confirmed the high efficacy of the novel DC vaccine in treating established tumors, also demonstrated the kinetics of tumor regression following the treatment (FIG. 2).

### Example 3

### The IL-10−/−DC Vaccine is Highly Effective in Triggering Protective Anti-Tumor Immunity Through Establishment of Immunological Memory

The IF-HCC model was also used to further study the immunological mechanism underlying the anti-tumor effect by pre-vaccination of the mice prior to tumor implantation.

Materials and Methods

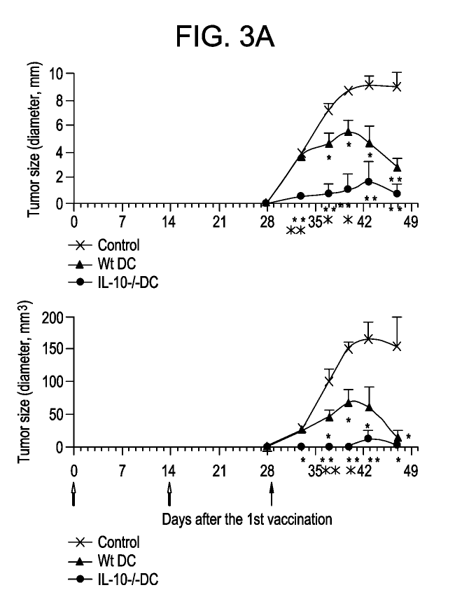

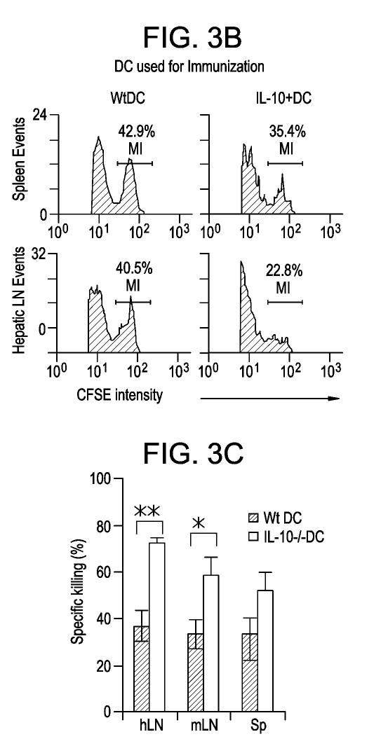

Groups of normal C57BL/6 mice (n=4) were first given 2 injections of the DC-vaccines at bi-weekly intervals (open arrows), followed by tumor implantation at day 28 (solid arrow). Tumor development was measured at daily intervals post-tumor implantation, and expressed as diameters (upper graph) and volume (lower graph) of the tumor mass (FIG. 3A).

In vivo Hepa 1-6 antigen-specific cytotoxic killing in lymphoid organs of the DC-vaccinated mice was subsequently evaluated. Normal naïve C57BL/6 mice were immunized twice as described above in (FIG. 3A). Two weeks after the second vaccination, the mice were injected with tumor-antigen-pulsed and CFSE-labeled target cells based on and modified from a protocol previously developed by Dercamp et al. Cancer Res., 65:8479-86 (2005). Forty eight hours after the target cell injection, the mice were killed and different lymphoid organs including spleens, hepatic lymph nodes (hLN) and mesenteric lymph nodes (mLN) removed for analysis by flow cytometry.

Representative FACS profiles show relative frequencies of the remaining tumor antigen-pulsed (CFSE^{hi}) and un-pulsed control (CFSE^{lo}) target cells detected in the spleen and hLN of the vaccinated mice (FIG. 3B). The inserted figure represents the absolute ratio of the tumor antigen-pulsed (CFSE^{hi}) over the total CFSE⁺ (CFSE^{hi}+CFSE^{lo}) target cells detected in the corresponding lymphoid organs. The percentage of tumor-specific killing (lysis) in the lymphoid organs of the vaccinated mice was then individually calculated and shown in (FIG. 3C). *depicts significant difference between the IL-10^{-/-}DC vaccine and the WtDC vaccine-treated groups, and *depicts significant difference between the IL-10−/−DC vaccine-treated group and the PBS-treated control group (* or *p<0.05, ** or **p<0.01, Student's T test).

Results

The mouse IL-10^{-/-}DC HCC vaccine was highly effective in triggering protective anti-tumor immunity (FIG. 3A), through the establishment of strong immunological memory and effective tumor specific T cell immunity. FIG. 3B-C shows the in vivo Hepa 1-6 antigen-specific cytotoxic killing in lymphoid organs of the DC-vaccinated mice.

### Example 4

### An Effective Vaccine Delivered by IL-10^{-/-}DC Evokes Protective Anti-Tumor Immunity Against Mouse Melanoma

The superiority of IL-10^{-/-}DC as the vector to deliver tumor vaccine was further tested and confirmed in a different tumor model, melanoma in mice. The vaccine delivery system is also effective in triggering protective immunity against the tumor. Mice pre-vaccinated with tumor antigen-loaded IL-10″-DC, but not conventional wild type DC (WtDC), were effectively protected from tumor development.

Materials and Methods

A mouse melanoma model previously developed by Kikuchi et al was adopted (Kikuchi, T., et al., Blood, 96(1):91-9 (2000)). Briefly, normal inbred C57BL/6 mice were injected into the left flank subcutaneously (s.c.) with different number of live melanoma (B16) tumor cells (0.01, 0.1 million cells per mouse). Tumor development was monitored and recorded daily.

The above model was then used to test further the DC vaccine. Briefly, normal naive C57BL/6 mice were first injected with DCs generated from the IL-10 knock-out (IL-10^{-/-}DC) or the wild type (WtDC) control mice that had been co-cultured overnight with necrotic B16 tumor cells.

In this study, necrotic tumor cells generated by freezing and thawing were used as the source of tumor antigens, and the DCs were found to be very effective in taking up the necrotic cells in vitro. One million DCs per mouse were injected i.v., and for two injections at 2-week intervals. Other control groups included mice injected with DC alone (without tumor cell co-culturing), and PBS only (non-vaccinated control). Two weeks after the second DC immunization, the mice were injected with live tumor cells (B16, 2×10⁴ cells/injection, left flank, s.c.), and the tumor development monitored.

Results

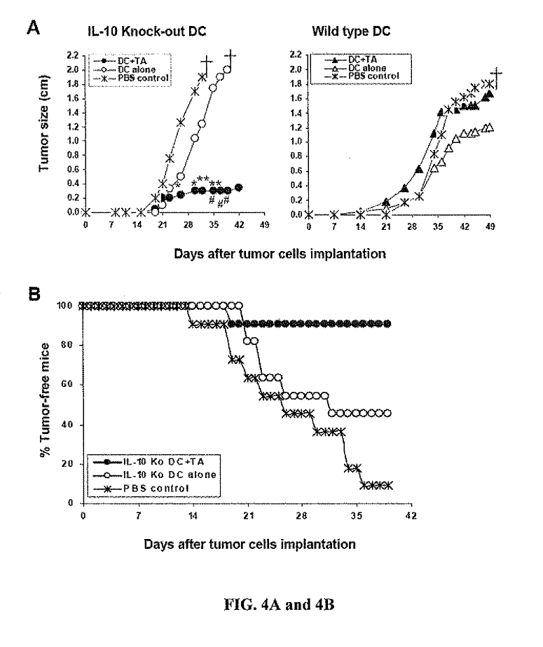

FIG. 4 shows that the mice pre-vaccinated with tumor antigen-loaded IL-10^{-/-}DC, but not conventional wild type DC (WtDC), are effectively protected from tumor development. This is evident in terms of both the average tumor size (FIG. 4A, n=6) and percentage of tumor-free mice (FIG. 4B, results combined from two experiments, n=11). The cross symbol indicates time point at which >50% of the mice were put down due to the development of tumor reaching the maximal size allowable (2.5 cm).

### Example 5

### Phenotypic and Functional Characterization of IL-10^{-/-}DC

### DCs Devoid of IL-10 Have a Highly Immunogenic Phenotype

Phenotypic analysis of the cells indicates that IL-10^{-/-}DC are of a highly immunogenic phenotype characterized by the expression of enhanced levels of MHC class II and CD 86, which are functional molecules known to be crucial for DC immunogenecity.

Materials and Methods

DCs were generated from mouse bone marrow precursors in the presence of GM-CSF, with (G4 DC) or without (GM DC) IL-4. At day 7, the DC phenotypes were determined by flow cytometry using specific antibodies to different DC phenotypic and functional markers.

Results

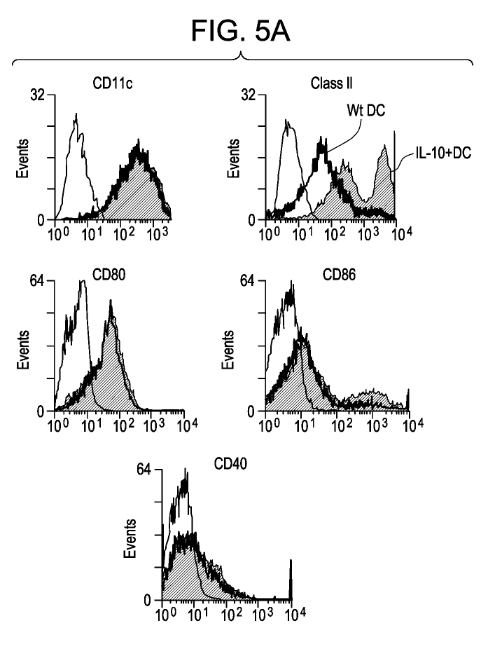

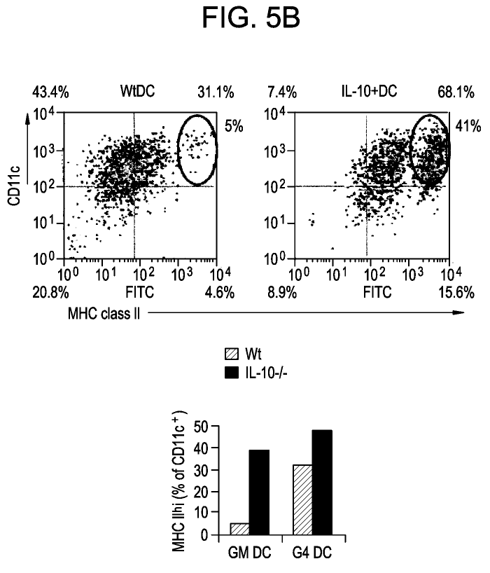

FIG. 5A. MHC class II, CD11c, CD80, CD86 and CD40 expression on IL-10^{-/-}DC (filled histogram) and WtDC (grey solid line), which were Day 7 DCs generated in the presence of GM-CSF alone. Dotted line: isotypic control. FIG. 5B. Frequency of DCs expressing high MHC class II molecules (MHC II DC) as determined by gating on CD11c⁺MHC^{hi} cells (oval gated region) and expressed as the percentage of total CD11c⁺ cells, and the bar histograms also compare the MHC II^{hi} cell frequencies for both the GM DCs and G4 DCs. The results showed that DCs generated from IL-10 knock-out mice expressed very high levels of MHC class II, a molecule known to be crucial for antigen presentation to CD4⁺ T helper cells. The IL-10^{-/-}DC also expressed enhanced level of CD86, an important co-stimulatory molecule required for the activation of naïve T-cells.

### Example 6

### Functional Characterization of IL-10^{-/-}DC (II)

### DCs Devoid of IL-10 Express Markedly Enhanced Levels of Th1-Type of Cytokines Known to be Crucial in Mediating Anti-Tumor Immunity

To understand the immunological mechanism underlying the superiority of the DC vector, cytokine expression profile of the IL-10^{-/-}DC was also analyzed.

Materials and Methods

DCs were generated from mouse bone marrow precursors in the presence of GM-CSF, with (G4 DC) or without (GM DC) IL-4 and, at day 6, the GM DC and G4 DC were stimulated with LPS (1 μg/ml, filled symbols), or cultured without stimulation (open symbols), for 12, 24 and 48 hours. Cytokine levels in the culture supernatants were measured by ELISAs specific for IL-10, IL-12, IFN-γ and TNF-α, respectively. Statistical significance of the differences between IL-10−/−DC and WtDC is indicated for both the LPS-induced (*) and spontaneous (*) cytokine release respectively (Student's T test, * or *p<0.05, ** or **p<0.01).

Results

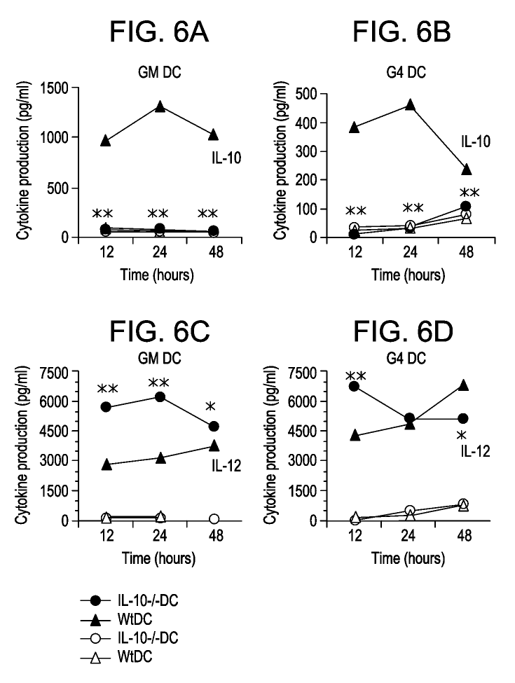

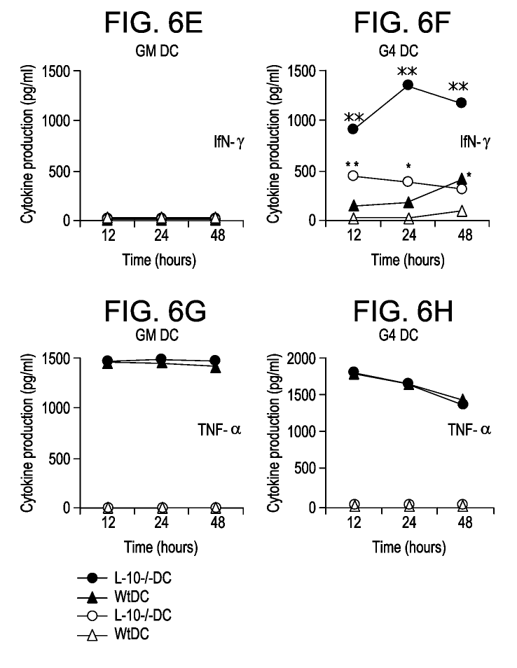

FIG. 6 shows that DCs devoid of IL-10 expressed enhanced levels of IL-12 and IFN-γ, two key Th1 or Th1 driving cytokines crucial in mediating anti-tumor immunity.

### Example 7

### Functional Characterization of IL-10^{-/-}DC (III)

### DCs Devoid of IL-10 are Highly Efficient Antigen Presenting Cells in Stimulating Allogeneic T Cell Responses

To evaluate the overall functional activities of IL-10^{-/-}DC, their ability for stimulating both syngeneic and allogeneic T-cell responses was measured.

Materials and Methods

Day 6 DCs generated from bone marrow precursors of the IL-10 knock-out (IL-10^{-/-}DC) and wild type control (WtDC) mice (both C57BL/6 background, H-2b), with (killed DC) or without (live DC) mytomycin-C treatment, were used as the APCs (effectors). Splenocytes from Balb/c (H-2d, allogenic), C57/BL6 (H-2b, syngenic) and the C57BL/6 IL-10−/− mice (H-2b, syngenic) were used as the responder cells, and added respectively at different effector to responder (DC:SPC) ratios.

Results

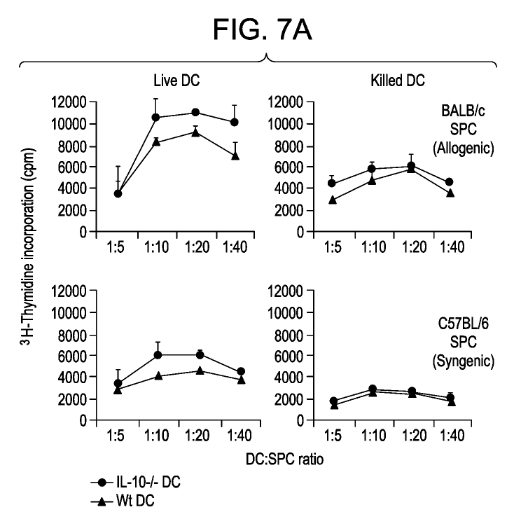

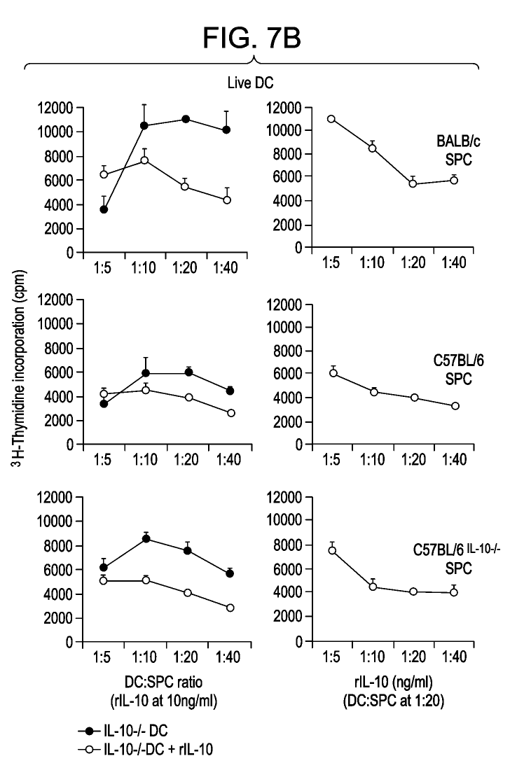

FIG. 7A is a comparison of allogenic and syngenic T responsiveness to live and killed (mitomycin-C treated) DCs. FIG. 7B shows the effects of recombinant IL-10 on the IL-10^{-/-}DC-mediated MLR responses. The effects of exogenous IL-10 were determined by adding a fixed amount (10 ng/ml) or serial concentrations (as indicated in the graph) of recombinant mouse IL-10 (R&D, 1023-ML-010) in the selected co-cultures of live IL-10^{-/-}DC and splenocytes from the 3 different mouse strains respectively. Cell proliferation was measured by H³-thymidine incorporation (0.5 μCi per well added for the last 8 hrs), and data shown at day 2. In summary, the results in FIG. 7 show that DCs devoid of IL-10 are superior to the conventional DCs (WtDC) as antigen presenting cells (APCs) for the induction of T-cell responses. More importantly, by the killing of DC cells, or by addition of exogenous IL-10, the role of DC-derived IL-10 in preventing the efficacy of conventional DC vaccines is confirmed.

### Example 8

### Generation of Human DCs Knockdown of IL-10 by siRNA

The findings in mouse models described above have provided a good basis for the technology to be translated directly into the development of novel tumor vaccines for clinical applications. In order to develop human DC-based anti-tumor vaccines with high efficacy for clinical and commercial applications, several approaches (operational strategies) are designed here (and below, see Example 9) for the generation of human DCs lacking or devoid of IL-10. The first approach is to generate human DCs knockdown of IL-10 expression by siRNA.

Materials and Methods

DCs are to be generated in vitro (outside the body), by established standard protocols (Caux, C. and B. Dubois, Methods in Molecular Medicine: Dendritic cell protocols, S. P. Robinson and A. J. Stagg, Editors. 2001, Humana Press: Totowa, N.J. p. 257-274; Fairchild, P. J., et al., Curr Biol., 10(23):1515-8 (2000)), from various types of DC precursors including patients' own (autologous) or donors' (allogenic) peripheral blood mononuclear cells (PBMC), and from bone marrow or embryonic stem cells.

Autologous DCs generated from patients' own peripheral blood mononuclear cells.

Autologous DCs generated from patients' own bone marrow cell precursors.

Allogeneic DCs generated from peripheral blood mononuclear cells of the MHC-matched blood donors.

Allogeneic DCs generated from bone marrow cell precursors of the MHC-matched blood donors.

Embryonic stem cell-derived DCs.

The discovery that 21-23 nucleotide RNA duplexes, known as small interfering RNAs (siRNAs) or RNA interference (RNAi), can knockdown the homologous mRNAs in mammalian cells has revolutionized many aspects of drug discovery (Singer, O., et al. Proc Natl Acad Sci USA., 101(15):5313-4 (2004)). The post-transcriptional knockdown via siRNA has now been proven to be the method of choice (quick and reliable) for studying molecular functions of cells.

The siRNA approach is therefore a desirable method for the selective inhibition of human interleukin-10 gene expression for the generation of novel DC vector cells, and for clinical therapeutic applications.

Two different methods for preparing human IL-10 siRNA are proposed: (1) The dicer approach, and (2) the pre-designed siRNA sequences:

The dicer approach includes a sequence-based analysis of the IL-10 gene to identify a suitable target sequence, the design of primers for a PCR-based amplification of the gene segment for random generation of the siRNA, and the introduction of d-siRNA into the target DCs or DC precursor cells.

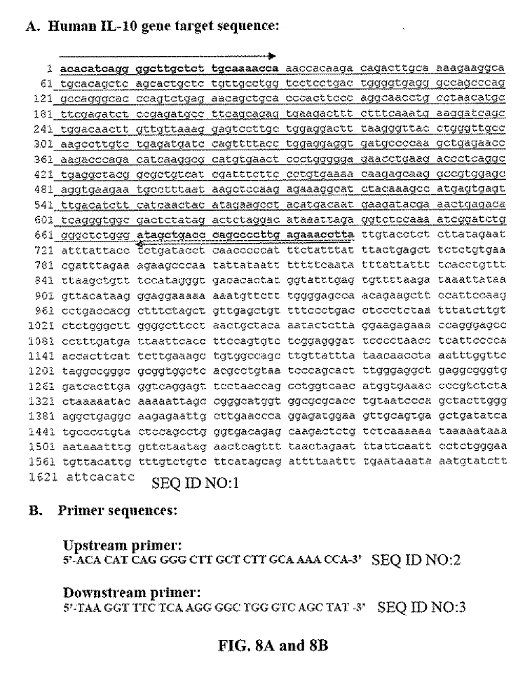

A fragment of 700 bp in length located at the 5, end of the human IL-10 gene is chosen as the target sequence (FIG. 8A, underlined). There is no significant sequence homology between this gene fragment and that of other known functional proteins. A pair of primers are then designed (FIG. 8B, and see below) for PCR amplification of this fragment using BLOCK-iT™ RNAi TOPO® Transcription Kit (Invitrogen), according to standard protocols provided by the manufacturer. Briefly, the 1 to 700 bp fragment of the TL-10 gene is amplified, by PCR using Taq polymerase and the specific primers:

IL-10-forward primer:

(SEQ ID NO: 2)

5′-ACA CAT CAG GGG CTT GCT CTT GCA AAA CCA-3′
IL-10-reverse primer:

(SEQ ID NO: 3)

5′-TAA GGT TTC TCA AGG GGC TGG GTC AGC TAT -3′.

The primary PCR products are then purified and linked to BLOCK-iT™ T7-TOPO® Linker which contains the sequence of a T7 promoter. The TOPO®-linked PCR products serve as templates in secondary PCR amplification using BLOCK-iT™ T7 primer in combination with each forward or reverse gene specific primer to produce sense and anti-sense DNA templates, respectively. Single strand sense and anti-sense RNA transcripts (ssRNA) are prepared by in vitro transcription with the T7-promoter-containing DNA templates from the secondary PCR. The dsRNAs are subsequently generated by annealing of the sense and anti-sense RNA transcripts and purified by using the BLOCK-iT™ RNAi Purification.

The small (21-23 nucleotides) double-stranded siRNA (d-siRNAs) are prepared from the dsRNAs and introduced into various types of target cells (see above), using BLOCK-iT™ Dicer RNAi Kit (Invitrogen) in accordance with the manufacturer's instruction. Briefly, the dsRNAs are first incubated with BLOCK-iT™ Dicer enzyme to produce d-siRNAs which are then purified using BLOCK-iT™ RNAi Purification Reagents. The size of purified d-siRNAs is checked by electrophoresis on an agarose gel. The concentration and purity of the d-siRNAs is then estimated using spectrometric method. Transfection of the purified d-siRNA into the cells can then be achieved using Lipofectamine™ 2000 reagent, to generate the DCs or DC precursors whose IL-10 gene is selectively blocked.

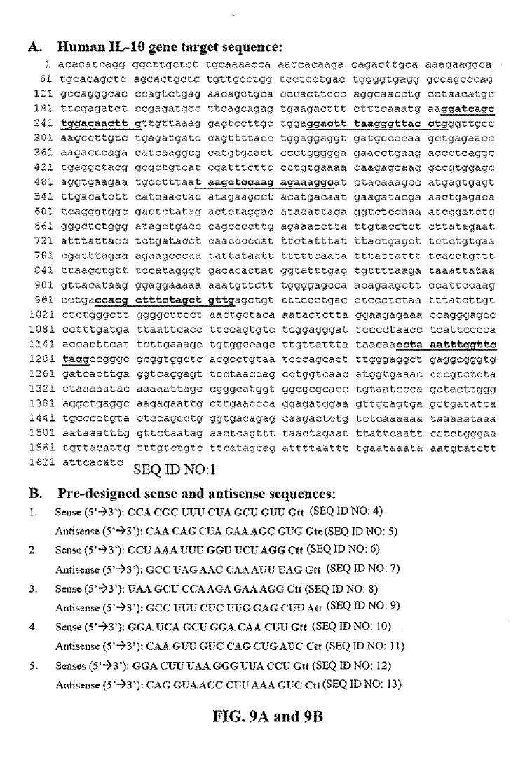

Another approach uses pre-designed human IL-10 siRNA sequences. This is an alternative method for the generation of human IL-10 siRNAs. The sequences of these small RNAs are pre-designed, according to the gene target sequence and standard selection criteria. FIG. 9A shows the positions on the human IL-10 gene corresponding to 6 sets of the proposed siRNA sequences (highlighted). The pre-designed human IL-10 siRNA sense and antisense sequences are given in FIG. 9B (synthesized by OriGene Rockville, Md. 20850, USA).

### Example 9

### Establishment of a Bank of Embryonic Stem Cell-Derived DCs Devoid of IL-10 (esDC^{IL10-/-})

This is another approach through the generation of DCs from human embryonic stem cell precursors (esDC) genetically programmed to delete the IL-10 encoding gene, in order to establish a bank of esDC devoid of IL-10 (esDC^{IL10-/-}). A major advantage of this approach is the unlimited supply of the cells, once the cell bank is established.

Materials and Methods

Generation of targeted ES cells devoid of IL-10 gene will be subjected to strict ethical approval. Human embryonic or trophoblastic stem cells, or cell lines derived from which, can be potentially used for the generation of human esDCs devoid of IL-10 in vitro.

To block IL-10 expression, the gene encoding human IL-10 precursor (ACCESSION NUMBER: NT_{—}021877 REGION: 459760.463559) will be deleted by homologous recombination. Briefly, for homologous recombination across the coding region of IL-10, regions 3′ and immediately 5′ of this region are PCR amplified. Primers are designed to incorporate restriction enzyme cleavage sites for ligation into the vector pSP:lacZ(NLS)loxNeo. The primer for the 5′ end of the 5′ arm introduces an AscI digestion site to allow linearization of the final construct. These PCR fragments are then ligated into the vector either side of a cassette encoding the genes for β-galactosidase and neomycin resistance. The linearized construct is then introduced into the ES cells by electroporation and transfected clones are selected in G418 (Invitrogen). ES cell clones are then screened for homologous recombination of the construct over the targeted region of endogenous IL-10 by Southern blot analysis of SpeI-digested genomic DNA with a specific PCR fragment.

The procedure for generation of human esDCs in vitro will be based on a protocol previously developed for mouse esDC by Fairchild et al (Fairchild, P. J., et al., Curr Biol, 10(23):1515-8 (2000)). Briefly, unmodified and the IL-10 gene targeted human ES cells are to be cultured in the presence of essential DC growth factors (cytokines: human GM-CSF and IL-3) for 7-10 days in completed culture medium at 37° C., 5% CO2. The culture medium is changed every 2-3 days with freshly supplemented cytokines.

### Example 10

### Phenotypic and Functional Characterization of the Human DCs Knockdown or Knockout of IL-10 Gene

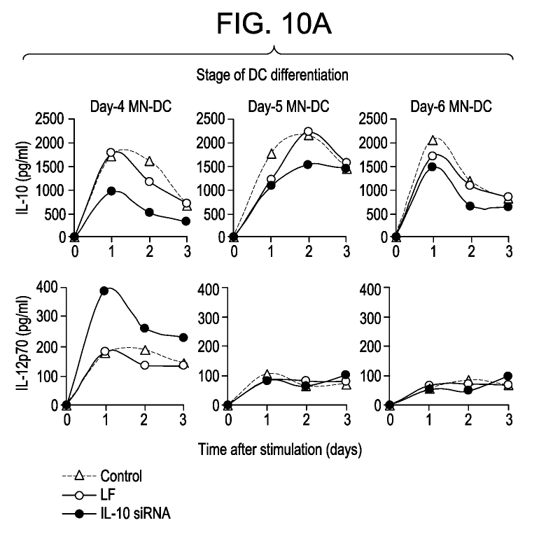

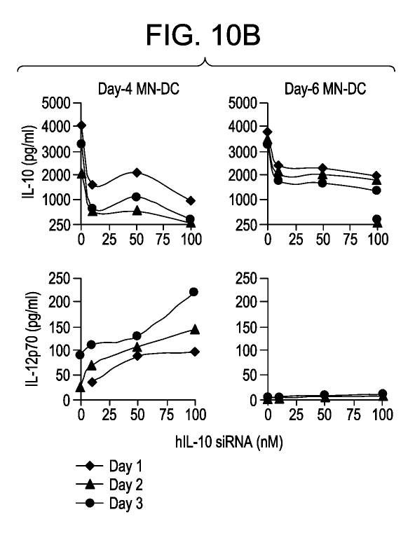

The effectiveness of IL-10 blockage is first assessed by RT-PCR for IL-10 mRNA expression in the cells. The ability of these cells to produce IL-10 is then determined at the protein level by specific ELISA immunoassays commercially available (R&D). A detailed kinetic study (time and dosage) of the IL-10 siRNA-treated human monocyte-derived DC (MN-DC) has been carried out to finely optimize the conditions. Satisfactory blockage of IL-10 expression and its associated enhancing effects on IL-12p70 production, by human MN-DCs have been observed and, under the optimized time and dose kinetic conditions, a full blocking of IL-10 expression is also achievable (see FIG. 10).

Before clinical application, the esDC^{IL-10-/-} cells will also be subjected to further functional characterizations, and classification including standard HLA (human leukocyte antigen) typing. The cells of major HLA types generated from different donors can then be sorted, classified and a full range of the esDC^{IL-10-/-} with specified HLA types can be made available anytime and continuously to the patients in need.

Both defined (e.g., specific protein antigens, or peptides derived from an identified tumor antigen) and undefined (e.g. tumor cell lysate, necrotic tumor cells) tumor antigens can be used for loading the DC vectors. The same procedure described above will be used for tumor antigen loading. Afterwards, the cells are washed and counted, and then re-suspended in sterile saline which will be ready for clinical applications.

For quality control, the functional phenotype of the various types of DCs generated as above after antigen loading will be further analyzed before therapeutic use. The immunogenicity of the IL-10−/−DC or esDC^{IL10-/-} are to be assessed by cell surface expression of key DC functional molecules (MHC class I and class II; CD80, CD86 and CD40), and by their expression levels of IL-10 versus those important Th1 cytokines (IL-12, IFN-γ and TNF-α) as described above.

### Example 11

### Generation of Rat DCs Knockdown of IL-10 by siRNA

In order to prepare for clinical application on cancer patients, the siRNA approach has been repeatedly tested in a rat HCC lung metastasis model, and subsequently rat orthotopic solid liver tumor (HCC) models (see Example 12 below), to evaluate the clinical efficacy of the IL-10 siRNA-treated DC vaccine. The two approaches and detailed experimental procedure described above for human IL-10 siRNA were similarly adopted for the rat system.

Materials and Methods

The potential IL-10 siRNA target sequence (position 1-624, underlined in FIG. 11A) is selected based on the rat IL-10 gene map, and for its lack of sequence homology with that of other known functional proteins. For PCR amplification of the selected gene segment nucleotide sequences of IL-10 primers corresponding to positions 18-37 and 600-624 (FIG. 11A, in bold) were designed and shown in (FIG. 11B).

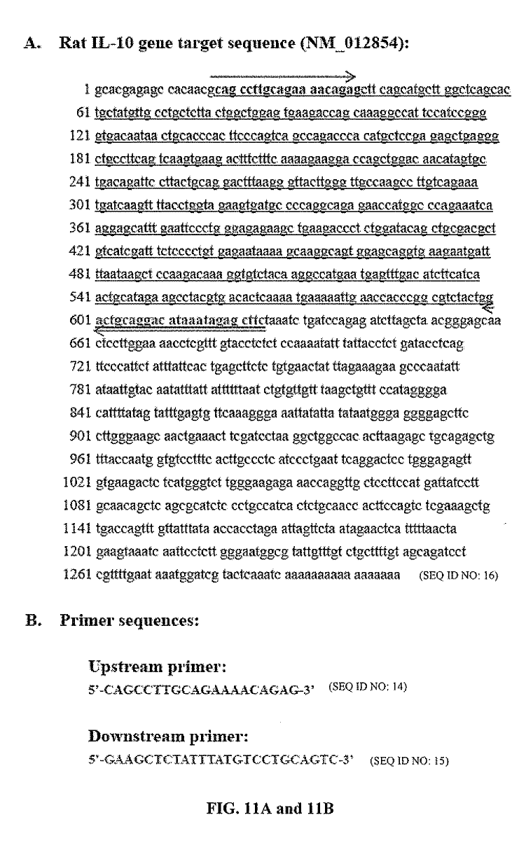

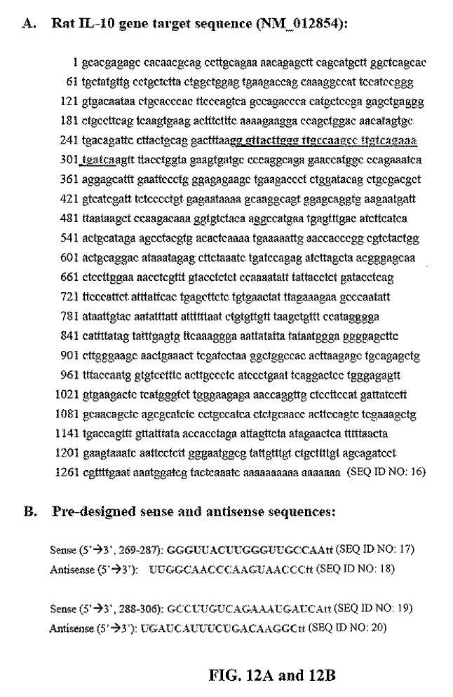

For the pre-designed siRNA approach, the rat IL-10 gene target sequence (positions corresponding to the two pre-designed siRNA sequences highlighted, FIG. 12A), and the pre-designed rat siRNA sense and antisense sequences (FIG. 12B) are shown.

The effectiveness of IL-10 blockage was assessed as described above similarly for the human siRNA-treated human DCs. In brief, DCs were generated from rat bone marrow precursors in the presence of GM-CSF for 7 days. At different DC differentiation and maturation stages (Day-3, 4, 5), IL-10 siRNA (100 nM in lipofectamine 2000, pre-designed sequences) was added. IL-10 production by the cells in response to LPS stimulation was determined by ELISA.

Results

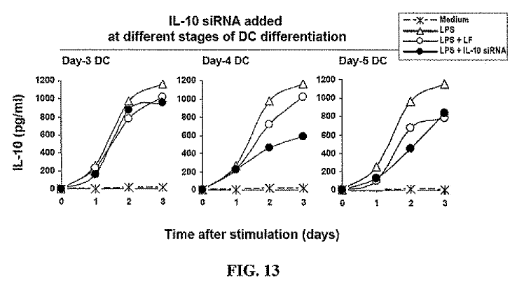

FIG. 13 shows the blocking efficiency of siRNA on IL-10 production by the rat BMDC at different maturation stages in cultures.

### Example 12

### Immunotherapy Against Cancer Cell Metastasis Using IL-10 siRNA-treated DC in a Rat HCC Lung Metastasis Model

Therapeutic anti-tumor immunity elicited by the IL10 siRNA-treated DC vaccine was evaluated in the rat HCC lung metastasis model, to examine its effects on post-operational tumor metastasis following surgical resection of the primary liver tumors.

Materials and Methods

The method of Man, K., et al., Liver Transpl., 13(12):1669-77 (2007) was used as follows. Normal Buffalo rats (male, 8-10 wks) were implanted into the liver (left lobe) surgically with an orthotopic liver tumor block (2-3 mm cube). The orthotopic tumors were pre-established in vivo in the same strain of rat by intra-hepatic injection of luciferase-labeled tumor cells (rat HCC cell line, CRL1601), and the tumor growth monitored by the Xenogen in vivo animal imaging system (Xenogen IVIS100, Xenogen, US). The tumor tissues were harvested 3 weeks after the injection, and cut into small blocks (2-3 m cube) for the subsequent surgical implantation. Around 3 weeks after the tumor implantation, when the tumor nodule reached 2 cm in diameter, a procedure of partial hepatic ischemia/reperfusion injury was carried out before hepatectomy was conducted to remove the tumor bearing lobe (left). Lung metastasis was monitored subsequently at weekly intervals by Xenogen IVIS-100 (Xenogen, US).

The model was subsequently used to evaluate the rat DC vaccine. Groups of normal Buffalo rats (male, 8-10 wks) were implanted into the liver (left lobe) surgically with an orthotopic liver tumor block as described above. Three weeks after the tumor implantation, a procedure of partial hepatic ischemia/reperfusion injury was carried out before hepatectomy was conducted to remove the primary tumor. The animals were subsequently injected i.v. with either PBS only (Untreated Control, n 5), or the tumor antigens (rat HCC CRL1601 cell lysate) loaded DCs which had been pre-treated with IL-10 siRNA (IL-10 siRNA DC, n=7) or lipofetamine only (Control DC, n=6). Lung metastasis was then monitored at weekly intervals by Xenogen IVIS-100 (Xenogen, US), and confirmed by histology.

Results

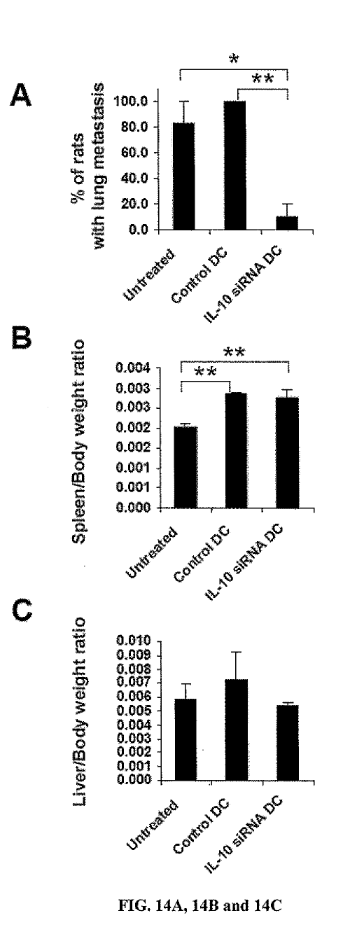

The percentage of rats developed lung metastasis (FIG. 14A), the Spleen/Body weight ratio (FIG. 14B), and the Liver/Body weight ratio (FIG. 14C) in the 3 experimental groups were recorded and compared. Data shown are results pooled from 2 repeated experiments (n=5˜7). Significant reduction in the cancer cell metastasis rate was observed in the IL-10 siRNA DC vaccine group as compared to the Control DC vaccine or PBS-treated group respectively. *p<0.05; **p<0.01 (Student t Test).

### Example 13

### Immunity Against Established Solid Tumors Induced by the IL10 siRNA-Treated DC Vaccine in the Rat Liver Orthotopic Tumor Model

Materials and Methods

Normal Buffalo rats (male, 8-10 wks) were injected i.v. for two times at bi-weekly intervals with the tumor antigens (rat HCC CRL1601 cell lysate) loaded DCs, either with (IL-10 siRNA DC) or without (Control DC) IL-10 siRNA pre-treatment, or injected with PBS only (Untreated control). Two weeks after the last immunization, the animals were implanted into the liver (left lobe) surgically with a pre-established orthotopic liver tumor (CRL1601) block as described above in Example 12, but without surgical removal of the primarily implanted tumor. Tumor growth was monitored at weekly intervals by the MRI scan.

Results

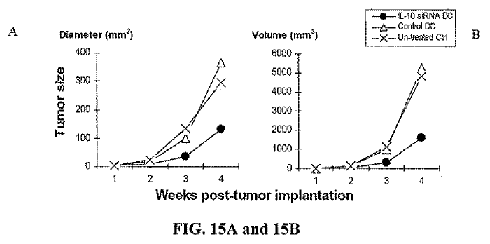

FIGS. 15A and 153B show the kinetics of tumor development in the vaccinated and non-vaccinated (Un-treated control) animals.

Modifications and variations will be obvious to those skilled in the art from the foregoing detailed description and are intended to come within the scope of the appended claims. References are specifically incorporated by reference.

What is claimed is:

1. A recombinant antigen presenting cell having reduced or inhibited immunosuppressive cytokine expression relative to a control.

2. The recombinant antigen presenting cell of claim 1 wherein the recombinant antigen presenting cell is a dendritic cell.

3. The recombinant antigen presenting cell of claim 1 wherein the recombinant antigen presenting cell comprises an inhibitory nucleic acid that inhibits or reduces expression of the cytokine in the recombinant antigen presenting cell.

4. The recombinant antigen presenting cell of claim 3 wherein the inhibitory nucleic acid binds to cytokine mRNA.

5. The recombinant antigen presenting cell of claim 3 wherein the inhibitory nucleic acid is selected from the group consisting of siRNA, antisense RNA, antisense DNA, and microRNA.

6. The recombinant antigen presenting cell of claim 1 wherein the cytokine is IL-10.

7. The recombinant antigen presenting cell of claim 1 wherein the cytokine is selected from the group consisting of TGF-β, IL-27, IL-35, indoleamine 2,3-dioxygenase or combinations thereof.

8. The recombinant antigen presenting cell of claim 1 wherein the recombinant antigen presenting cell comprises an antigenic polypeptide or antigenic peptides in complexes with MHC class I and MHC class II proteins.

9. The recombinant antigen presenting cell of claim 1 wherein the antigenic polypeptide is selected from the group consisting of tumor specific antigens, viral antigens, bacterial antigens, protozoan antigens, antigenic fragments thereof and combinations thereof.

10. A cell-based anti-tumor vaccine comprising dendritic cells (DCs) genetically modified to inhibit or block expression of IL-10.

11. The vaccine of claim 10, wherein IL-10 expression is transiently blocked by small interfering RNA (siRNA).

12. The vaccine of claim 10, wherein IL-10 expression is permanently blocked by deletion of the IL-10 gene through homologous recombination.

13. The vaccine of claim 10, wherein the dendritic cells are selected from the group consisting of cells derived from a patient's peripheral blood mononuclear cells, cells derived from a patient's bone marrow cell precursors, cells derived from MHC-matched donors' peripheral blood mononuclear cells, cells derived from MHC-matched donors' bone marrow cell precursors, and cells derived from MHC-matched donors' embryonic stem cells.

14. The vaccine of claim 10, wherein the dendritic cells are further genetically modified to have reduced expression of an additional immunosuppressive cytokine(s) or a tolerogenic molecule(s).

15. The vaccine of claim 14, wherein the additional immunosuppressive cytokine is selected from the group consisting of TGF-β, IL-27, and IL-35.

16. The vaccine of claim 14, wherein the tolerogenic molecule is indoleamine 2,3-dioxygenase.

17. A method for inducing tumors in a mammal comprising injecting tumor cells through the mammal's portal vein.

18. A cell bank comprising MHC-typed dendritic cells having a deletion of the gene for IL-10.

19. A method for inducing an immune response in a subject comprising administering the recombinant antigen presenting cell of claim 1 to the subject.

20. A method for immunotherapy, comprising administering a molecular or pharmacological blockage of interleukin 10 (IL-10) production, IL-10 activities or IL-10 gene expression in vitro or in vivo.

21. The method of claim 20 further comprising administering before, simultaneously, or after the immunotherapy, (chemotherapy, radiotherapy, or surgical resection of primary tumors.


SEQUENCE LISTING (20 sequences)

SEQ ID NO: 1 - DNA, 1629 bp, Homo sapiens
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 2 - DNA, 30 bp, Artificial Sequence
  Synthetic IL-10 forward primer
acacatcaggggcttgctcttgcaaaacca

SEQ ID NO: 3 - DNA, 30 bp, Artificial Sequence
  Synthetic IL-10 reverse primer
taaggtttctcaaggggctgggtcagctat

SEQ ID NO: 4 - DNA, 21 bp, Artificial Sequence
  SYnthetic predesigned SiRNA sense sequence
ccacgcuuucuagcuguugtt

SEQ ID NO: 5 - DNA, 21 bp, Artificial Sequence
  Synthetic pre-designed SiRNA antisense sequence
caacagcuagaaagcguggtc

SEQ ID NO: 6 - DNA, 21 bp, Artificial Sequence
  Synthetic predesigned SiRNA sense sequence
ccuaaauuugguucuaggctt

SEQ ID NO: 7 - DNA, 21 bp, Artificial Sequence
  Synthetic pre-designed SiRNA antisense sequence
gccuagaaccaaauuuaggtt

SEQ ID NO: 8 - DNA, 21 bp, Artificial Sequence
  Synthetic pre-designed SiRNA sense sequence
uaagcuccaagagaaaggctt

SEQ ID NO: 9 - DNA, 21 bp, Artificial Sequence
  Synthetic pre-designed SiRNA antisense sequence
gccuuucucuuggagcuuatt

SEQ ID NO: 10 - DNA, 21 bp, Artificial Sequence
  Synthetic pre-designed SiRNA sense sequence
ggaucagcuggacaacuugtt

SEQ ID NO: 11 - DNA, 21 bp, Artificial Sequence
  Synthetic pre-designed SiRNA antisense sequence
caaguuguccagcugaucctt

SEQ ID NO: 12 - DNA, 18 bp, Artificial Aequence
  Synthetic pre-designed SiRNA sense sequence
ggacuuuaaggguuaccu

SEQ ID NO: 13 - DNA, 21 bp, Artificial Sequence
  Synthetic pre-designed SiRNA antisense sequence
cagguaacccuuaaagucctt

SEQ ID NO: 14 - DNA, 20 bp, Artificial Sequence
  Synthetic Upstream primer
cagccttgcagaaaacagag

SEQ ID NO: 15 - DNA, 25 bp, Artificial Sequence
  Synthetic Downstream primer
gaagctctatttatgtcctgcagtc

SEQ ID NO: 16 - DNA, 1307 bp, Ratus ratus
gcacgagagccacaacgcagccttgcagaaaacagagcttcagcatgcttggctcagcactgctatgttgcctgctcttactggctggagtgaagaccagcaaaggccattccatccggggtgacaataactgcacccacttcccagtcagccagacccacatgctccgagagctgagggctgccttcagtcaagtgaagactttctttcaaaagaaggaccagctggacaacatagtgctgacagattccttactgcaggactttaagggttacttgggttgccaagccttgtcagaaatgatcaagttttacctggtagaagtgatgccccaggcagagaaccatggcccagaaatcaaggagcatttgaattccctgggagagaagctgaagaccctctggatacagctgcgacgctgtcatcgatttctcccctgtgagaataaaagcaaggcagtggagcaggtgaagaatgattttaataagctccaagacaaaggtgtctacaaggccatgaatgagtttgacatcttcatcaactgcatagaagcctacgtgacactcaaaatgaaaaattgaaccacccggcgtctactggactgcaggacataaatagagcttctaaatctgatccagagatcttagctaacgggagcaactccttggaaaacctcgtttgtacctctctccaaaatatttattacctctgatacctcagttcccattctatttattcactgagcttctctgtgaactatttagaaagaagcccaatattataattgtacaatatttattattttttaatctgtgttgtttaagctgtttccataggggacattttatagtatttgagtgttcaaagggaaattatattatataatgggaggggagcttccttgggaagcaactgaaacttcgatcctaaggctggccacacttaagagctgcagagctgtttaccaatggtgtcctttcacttgccctcatccctgaattcaggactcctgggagagttgtgaagactctcatgggtcttgggaagagaaaccaggttgctccttccatgattatccttgcaacagctcagcgcatctccctgccatcactctgcaaccacttccagtctcgaaagctgtgaccagtttgttatttataaccacctagaattagttctaatagaactcatttttaactagaagtaaatcaattcctcttgggaatggcgtattgtttgtctgcttttgtagcagatcctcgttttgaataaatggatcgtactcaaatcaaaaaaaaaaaaaaaaa

SEQ ID NO: 17 - DNA, 21 bp, Artificial Sequence
  Synthetic pre-designed SiRNA sense sequence
ggguuacuuggguugccaatt

SEQ ID NO: 18 - DNA, 21 bp, Artificial Sequence
  Synthetic pre-desinged SiRNA antisense sequence
uuggcaacccaaguaaccctt

SEQ ID NO: 19 - DNA, 21 bp, Artificial Sequence
  Synthetic pre-designed SiRNA sense sequence
gccuugucagaaaugaucatt

SEQ ID NO: 20 - DNA, 21 bp, Artificial Sequence
  Synthetic pre-designed SiRNA antisense sequence
ugaucauuucugacaaggctt